(19) United States
(12) Patent Application Publication
(10) Pub. No.: US 2012/0231470 A1
(43) Pub. Date: Sep. 13, 2012

(54) METHODS AND SYSTEMS ASSOCIATED WITH DETECTION OF FATTY ACID ELONGATION IN A CELL

(76) Inventors: Janek SZYCHOWSKI, MONTREAL (CA); David A. TIRRELL, PASADENA, CA (US); Sungjin Park, Pasadena, CA (US)

(21) Appl. No.: 13/414,670

(22) Filed: Mar. 7, 2012

Related U.S. Application Data
(60) Provisional application No. 61/449,995, filed on Mar. 7, 2011.

(51) Int. Cl. G01N 33/53; C12Q 1/02

(52) U.S. Cl. 435/7.1; 435/29

(57) ABSTRACT

Methods and systems to identify compounds capable of altering a fatty acid elongation pathway and for identifying conditions under which fatty acids elongation can occur in a cell are described. The methods and systems comprise labeled fatty acid precursors and cells capable of elongating fatty acids. Methods for providing suitable components of an assay for identifying compounds capable of altering a fatty acid elongation pathway are described.

### CROSS REFERENCE TO RELATED APPLICATIONS

The present application claims priority to U.S. Provisional Application No. 61/449,995 filed on Mar. 7, 2011 which is incorporated herein by reference in its entirety.

### STATEMENT OF GOVERNMENT FUNDING

This invention was made with government support under GM62523 awarded by the National Institute of Health. The government has certain rights in the invention.

### FIELD

The present disclosure relates to methods and systems associated to detection of fatty acid elongation in a cell. In particular, the present disclosure related to methods and systems and to related compositions for identification of compounds suitable to affect fatty acid biosynthesis.

### BACKGROUND

Fatty acid biosynthesis has been subject of several studies in view of the central role that the relevant pathway has been recognized for various processes of interest.

For example, fatty acidy biosynthesis has been identified as a validated cellular target for antibiotic development (Ref. 2). Also, fatty acid elongation pathway has been determined to be relevant for identification of compounds suitable for treatment of diseases and metabolic disorders such as certain cancers and obesity. (Ref. 3)

Current methods related to fatty acid biosynthesis can be implemented to detect viable candidate compounds able to affect fatty acid biosynthesis. However, applications and methods associated to a fatty acid elongation pathway such as high throughput screening and selection of compounds that affect and in particular inhibit fatty acid biosynthesis are still challenging in particular with reference to fatty acid biosynthesis in a cellular environment.

### SUMMARY

Provided herein are methods and systems and related compositions that in several embodiments allow performance of cell-based assays and in particular of cell based high throughput assays related to fatty acid elongation pathways.

According to a first aspect, a method and system are herein described that allow identification of a compound capable of altering a fatty acid elongation pathway. The method comprises contacting a candidate compound and a labeled fatty acid precursor with a cell comprising enzymes enabling the fatty acid elongation pathway, for a time and under condition to allow elongation of the labeled fatty acid precursor through the fatty acid elongation pathway and to allow interference of the candidate compound with the fatty acid elongation pathway. The method further comprising detecting fatty acid elongation through detection of the label following the contacting. The system comprises at least two of a fatty acid precursor, a label and reagents for detection of the label for simultaneous combined or sequential use in methods herein described. In some embodiments the fatty acid precursor is a labeled fatty acid precursor.

According to a second aspect, a method and system are described herein that allow determination of an effective concentration of one or more compounds capable of altering a fatty acid elongation pathway. The method comprises contacting the one or more compounds at a first concentration and a labeled fatty acid precursor with a cell comprising the enzymes required in the fatty acid elongation pathway, the contacting performed for a time and under condition to allow a first elongation of the labeled fatty acid precursor through the fatty acid elongation pathway and to allow interference of the one or more compound at the first concentration with the fatty acid elongation pathway. The method further comprises detecting a first labeling signal associated with the labeled fatty acid precursor following the first elongation, the first labeling signal associated with the first concentration, the detecting performed to determine concentration effective for altering a fatty acid elongation pathway. The system comprises at least two of a label, a fatty acid precursor, the one or more compounds in one or more compositions each comprising the compound at a concentration and reagents to detect the label for simultaneous combined or sequential use in methods herein described. In some embodiments the fatty acid precursor is a labeled fatty acid precursor

According to a third aspect, a method and system are described that allow identification of a cell capable of elongating an exogenous fatty acid. The method comprises contacting a candidate cell and a fatty acid precursor comprising a label, with a compound capable of altering a fatty acid elongation pathway at a concentration suitable for altering the fatty acid elongation pathway, the contacting performed for a time and under condition to allow elongation of the labeled fatty acid precursor through the fatty acid elongation pathway. The method further comprises detecting fatty acid elongation through detection of the label following the contacting. The system comprises at least two of a label, a fatty acid precursor, and reagents to detect the label for simultaneous combined or sequential use in methods herein described. In some embodiments the fatty acid precursor is a labeled fatty acid precursor

According to a fourth aspect, a method and system are described for identifying a compound capable of inducing apoptosis in a cancer cell is described. The method comprises identifying a compound capable of inhibiting a fatty acid elongation pathway according to methods herein described. The method further comprises contacting the identified compound with the cell for a time and under conditions to allow interference of the compound with the fatty acid elongation pathway; and detecting viability of the cell following the contacting. The system comprises at least two of a label, a fatty acid precursor, and the one or more compound in one or more composition each comprising the compound at a concentration, reagents to detect the label and reagents to detect viability of the cell for simultaneous combined or sequential use in methods herein described. In some embodiments the fatty acid precursor is a labeled fatty acid precursor

According to a fifth aspect, a method for identifying a value or a range of values of a parameter under which a cell is capable of elongating an exogenous fatty acid is described. The method comprises contacting the cell under a first value of the parameter, and a fatty acid precursor comprising a label, the contacting performed for a time and under condition to allow elongation of the labeled fatty acid precursor through the fatty acid elongation pathway. The method further comprises contacting the cell at one or more further values of the parameter and a fatty acid precursor comprising a label, the contacting performed for a time and under condition to allow elongation of the labeled fatty acid precursor through the fatty acid elongation pathway. The method further comprises detecting the label in the cell following the contacting and comparing detection signals of each condition of the cell. The system comprises at least two of a label, a fatty acid precursor, and reagents to detect the label for simultaneous combined or sequential use in methods herein described. In some embodiments the fatty acid precursor is a labeled fatty acid precursor

According to a further aspect a system is described for detection of inhibitors of a fatty acid elongation pathway. The system comprises at least two of a labeled fatty acid precursor, a cell and reagents to detect a signal from the labeled fatty acid precursor. In particular in several embodiments the labeled fatty acid precursor, the cell and the reagents to detect the signal from the labeled fatty acid precursor are for simultaneous combined or sequential use in the methods herein described.

Methods and systems herein described and related compositions, allow in several embodiments, screening and selection of compounds able to affect a fatty acid biosynthesis pathway in a cell. In some embodiments in a high throughput fashion.

Methods and systems herein described and related compositions, allow in several embodiments, screening and selection of cells able to elongate fatty acids which can be used in the methods and systems for screening and selection of compounds able to affect fatty acid biosynthesis, and in particular cells able to elongate exogenous fatty acids.

Methods and systems herein described and related compositions, allow in several embodiments, screening and selection of labeled fatty acid precursors which can be used in the methods and systems for screening and selection of compounds able to affect fatty acid biosynthesis, and in particular cells able to elongate exogenous fatty acids.

Methods and systems herein described and related compositions, allow in several embodiments a determination of a concentration of a compound effective for altering a fatty acid elongation pathway.

Methods and systems herein described and related compositions, allow in several embodiments, identification of a compound capable of inducing apoptosis in a cancer cell, particularly in combination with further tests to confirm apoptotic activity.

Methods and system herein described and related compositions, allow in several embodiments, identification of a condition under which a cell is capable of elongating a fatty acid, and in particular, an exogenous fatty acid.

The methods and systems herein described can be used in connection with medical, pharmaceutical, veterinary applications as well as fundamental biological studies and various applications, identifiable by a skilled person upon reading of the present disclosure, wherein detection of compounds able to affect a fatty acid elongation pathway is desirable. Exemplary applications for detection of fatty acid elongation inhibitors comprise drug research and other scientific research. Exemplary applications for compounds capable of enhancing a fatty acid elongation pathway comprise biofuel production.

The details of one or more embodiments of the disclosure are set forth in the accompanying drawings and the description below. Other features, objects, and advantages will be apparent from the description and drawings, and from the claims.

### BRIEF DESCRIPTION OF DRAWINGS

The accompanying drawings, which are incorporated into and constitute a part of this specification, illustrate one or more embodiments of the present disclosure and, together with the description of example embodiments, serve to explain the principles and implementations of the disclosure.

FIG. 1 shows a scheme depicting fatty acid elongation in a bacterial cell wherein R is a saturated or unsaturated aliphatic chain of various lengths and configurations.

FIG. 2 shows a general schematic of the high-throughput screening for fatty acid biosynthesis inhibitors and a structure of a cyclooctyne fluorescent dye.

FIG. 3 shows a diagram illustrating results of a high-throughput screening for fatty acid biosynthesis inhibitors. The y-axis is a measurement of the fluorescence intensity detected. The left pair of bars show the fluorescence intensity of the wells containing bacterial cells and no azido fatty acid or fatty acid biosynthesis inhibitor, the center pair of bars show the fluorescence intensity of the wells containing bacterial cells and only 6-azidohexanoic acid, and the right pair of bars show the fluorescence intensity of the wells containing bacterial cells and a mixture of 6-azidohexanoic acid and known fatty acid biosynthesis inhibitor cerulenin. The light grey bars represent a result of an assay with V. harveyi B392 strain bacteria and the dark grey bars represent results of an assay with V. harveyi CY1723 bacteria.

FIG. 4 show flow cytometry fluorescence histograms of the demonstration of Example 1 using V. Harveyi B392 and V. harveyi CY1723 bacteria. Panel A (left) shows the flow cytometry fluorescence histograms for V. harveyi B392, were the 18907 curve is the fluorescence histogram of the B392 bacteria treated with only 6-azidohexanoic acid, the 2347 curve is the fluorescence histogram of the B392 bacteria treated with a mixture of 6-azidohexanoic acid and known fatty acid biosynthesis inhibitor cerulenin, and the 1142 curve is the fluorescence histogram of the B392 bacteria treated with neither 6-azidohexanoic acid orcerulenin. Panel B (Right) shows the flow cytometry fluorescence histograms for V. harveyi CY1723, were the 4531 curve is the fluorescence histogram of the CY1723 bacteria treated with only 6-azidohexanoic acid and the 1704 curve is the fluorescence histogram of the CY1723 bacteria treated with a mixture of 6-azidohexanoic acid and known fatty acid biosynthesis inhibitor cerulenin.

FIG. 5 shows a schematic of a method for screening fatty acid biosynthesis inhibitors by measuring the fluorescence of a particular well relative to a control well.

FIG. 6 shows the fluorescence measurements of a high-throughput screening for fatty acid biosynthesis inhibitors using various commercially available fatty acid biosynthesis inhibitors, including cerulenin (Ref. 8), bischloroanthrabenzoxocinone (Ref. 10), thiolactomycin (Ref. 11), and platensimycin (Ref. 2) as well as a number of arbitrarily chosen drugs (listed in TABLE 3). The y-axis is the fluorescence ratio of a particular well relative to the control well with no antibiotic treatment. Columns of a well plate are organized from 1 to 12 on the x-axis and the rows of each column from A to H are arranged from left to right and denoted by corresponding shades of grey in the legend. Row A, Column 1 to 3 (cerulenin), Row H, Column 1 to 3 (thiolactomycin), and Row H, Column 3 to 6 (platensimycin) contain fatty acid biosynthesis inhibitors, while other wells correspond to non-fatty acid biosynthesis inhibitors as controls.

FIG. 7 shows the structures of some w-azido fatty acids produced by the elongation of 6-azidohexanoic acid by B392 cells.

FIG. 8 shows an HPLC-UV-VIS spectrum for a 1:1:1:1 mixture of analytical standards of the fatty acids of FIG. 7 after reaction with 5-hexyn-1-ol (structure of A, B, C and D are shown in FIG. 10). Peak A is the 10-carbon fatty acid, peak B is the 12-carbon fatty acid, peak C is the 14 carbon fatty acid, and peak D is the 16 carbon acid.

FIG. 9 shows an HPLC-UV-VIS spectrum of a saponified lipid extract of V. harveyi B392 cells fed with 6-azidohexanoic acid after reaction with 5-hexyn-1-ol. Peak A is the 10-carbon fatty acid triazole derivative, peak B is the 12-carbon fatty acid triazole derivative, peak C is the 14-carbon fatty acid triazole derivative, peak D is the 16-carbon saturated fatty acid triazole derivative, and peak E is the 16-carbon fatty acid triazole derivative.

FIG. 10 shows the chemical structures of fatty acid triazole derivatives. Labels A-E correspond to the peak letters in FIG. 9. For structure E, the localization and stereochemistry of the unsaturation is unknown.

FIG. 11 shows a mass spectrum of peak A in the spectrum of FIG. 9 depicting the detection of the 10-carbon fatty acid triazole derivative.

FIG. 12 shows a mass spectrum of peak B in the spectrum of FIG. 9 depicting the detection of the 12-carbon fatty acid triazole derivative.

FIG. 13 shows a mass spectrum of peak C in the spectrum of FIG. 9 depicting the detection of the 14-carbon fatty acid triazole derivative.

FIG. 14 shows a mass spectrum of peak D in the spectrum of FIG. 9 depicting the detection of the 16-carbon saturated fatty acid triazole derivative.

FIG. 15 shows a mass spectrum of peak E in the spectrum of FIG. 9 depicting the detection of the 16-carbon unsaturated fatty acid triazole derivative.

### DETAILED DESCRIPTION

Methods and systems are described herein that in several embodiments, allow assays associated to fatty acid biosynthesis in a cell

The term “fatty acid” as used herein indicates a carboxylic acid with an aliphatic tail (chain) of various lengths which is either saturated or unsaturated. Fatty acids in the sense of the present disclosure comprise short-chain fatty acids (SCFA) which are fatty acids with aliphatic tails of fewer than six carbons (e.g. butyric acid), medium-chain fatty acid (MCFA) which are fatty acids with aliphatic tails of 6 to 12 carbons, long-chain fatty acid (LCFA) which are fatty acids with aliphatic tails longer than 12 carbons, and very long chain fatty acid (VLCFA) which are fatty acids with aliphatic tails longer than 22 carbons. Most naturally occurring fatty acids have a chain of an even number of carbon atoms, from 4 to 28. Fatty acids in the sense of the present disclosure further comprise unsaturated fatty acids of including one or more double bonds in cis and/or trans configurations in various positions of the chain, or saturated fatty acids which usually have between 12 and 24 carbon atoms and have no double bonds. Fatty acids are usually derived from triglycerides or phospholipids. When they are not attached to other molecules, they are known as “free” fatty acids. Exemplary fatty acids comprise myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, caprylic acid, capric acid, lauric acid, myristic acid, palmitic acid, stearic acid, arachidic acid, behenic acid, lignoceric acid, cerotic acid

The term “fatty acid elongation pathway” indicates a process for the synthesis, and in particular biosynthesis, of a fatty acid. In particular, the term “pathway” as used herein indicates series of chemical reactions typically occurring within a cell. In each pathway, a principal chemical is modified by a series of chemical reactions, typically through a step-by-step modification of an initial molecule to form another product. Enzymes typically catalyze these reactions, and often require dietary minerals, vitamins, and other cofactors in order to function properly. Because of the many chemicals (e.g. “metabolites”) that may be involved, metabolic pathways can be elaborate. In addition, numerous distinct pathways can co-exist within a cell.

The term “biosynthesis” indicates an enzyme-catalyzed process in cells of living organisms by which substrates are converted to more products. A biosynthesis process often consists of a pathway including several enzymatic steps in which the product of one step is used as substrate in the following step. Examples for such multi-step biosynthetic pathways are those for the production of amino acids, fatty acids, and natural products. Biosynthesis typically plays a major role in all cells, and many dedicated metabolic routes combined constitute general metabolism. The prerequisites for biosynthesis are precursor compounds, chemical energy (such as in the form ATP), and catalytic enzymes, which may require reduction equivalents (e.g., in the form of NADH, NADPH). In particular, in several embodiments, biosynthesis of fatty acids involves the condensation of acetyl-CoA. Since this coenzyme carries a two-carbon-atom group, several natural fatty acids and in particular fatty acids that can be identified in a cell have even numbers of carbon atoms.

Exemplary fatty acid elongation pathways in particular directed to biosynthesis of fatty acids comprise saturated straight chain fatty acid elongation, unsaturated fatty acid elongation and branched-chain fatty acid elongation.

In particular, in an exemplary pathway directed to straight chain fatty acid elongation, a fatty acid precursor (for example, acetyl-coenzyme A) and malonyl-coenzyme A are typically reacted through a decarboxylative Claisen condensation catalyzed by a ketoacyl synthase enzyme to generate an intermediate that is two carbons longer than the original fatty acid precursor. Then through a series of protein-mediated and enzyme-catalyzed reactions, the intermediate is reduced to become a saturated elongated fatty acid intermediate. This intermediate is then elongated by two carbons by reaction with another molecule of malonyl-coenzyme A and repetition of the above enzyme catalyzed reduction reactions. The process repeats until a saturated fatty acid of necessary length (for example, 16 carbons) is made. Examples of enzymes and proteins involved in the saturated straight chain fatty acid elongation pathways include, but are not limited to, acetylCoA carboxylase, β-ketoacyl-ACP synthase, malonyl/acetyl-ACP transferases, 3-hydroxyacyl-ACP dehydrase, enoyl-ACP reductase, 3-ketoacyl-ACP reductase, acyl carrier protein, and thioesterase. Enzymes involved in the saturated straight chain fatty acid elongation pathways may be discrete and monofunctional (such as in Type II fatty acid synthase systems) or part of a larger multifunctional polypeptide (such as in Type I fatty acid synthase systems).

Exemplary fatty acid elongation pathways in particular directed to biosynthesis of unsaturated fatty acid comprise anaerobic and aerobic pathways. In the anaerobic pathway, enzymes (rather than oxygen) are typically responsible for the insertion of the double bonds (unsaturations). In one example of an anaerobic pathway, an intermediate in the normal saturated fatty acid elongation pathway (for example, β-hydroxydecanoyl-acyl carrier protein) is intercepted, and a series of enzyme cause the formation and isomerization of a carbon-carbon double bond which is then retained during the normal fatty acid elongation reactions described for saturated fatty acid elongation. One fatty acid product than can be made by this pathway is palmitoleic acid. In contrast, the aerobic pathway relies on oxygen as well as enzymes to generate the carbon-carbon double bonds. In this case the enzymes typically remove two adjacent hydrogens from a saturated fatty acid elongation intermediate or product. One example of such a transformation is the elongation of oleic acid from stearic acid. Examples of enzymes involved in the unsaturated fatty acid elongation pathways comprise, β-hydroxydecanoyl-ACP dehydrase (such as FabA), β-ketoacyl-ACP synthase (such as FabB), fatty acid desaturases (including, but not limited to, human fatty acid desaturases, bacterial fatty acid desaturases, plant stearoyl-acyl-carrier-protein desaturase, cyanobacterial DesA, and stearoyl-CoA desaturase-1), and cytochrome B5 reductase. In some instances an elongated fatty acids can be diverted from the elongation pathway to be desaturated and result in the formation of unsaturated fatty acid through a pathway that is also comprised in the scope of the present disclosure.

In branched-chain fatty acid elongation, branched chain (typically branching near the non-carboxyl end) fatty acids are typically synthesized via enzyme-catalyzed reactions from branched precursors using reactions similar to those used in saturated fatty acid elongation. One example of a branched fatty acid synthesized in this manner is 14-methylpentadecanoic (isopalmitic) acid. Examples of enzymes involved in the branched-chain fatty acid elongation pathways include, but are not limited to, branched-chain α-keto acid decarboxylase, transaminase, malonyl-CoA fatty acid synthase, and fatty acid synthase.

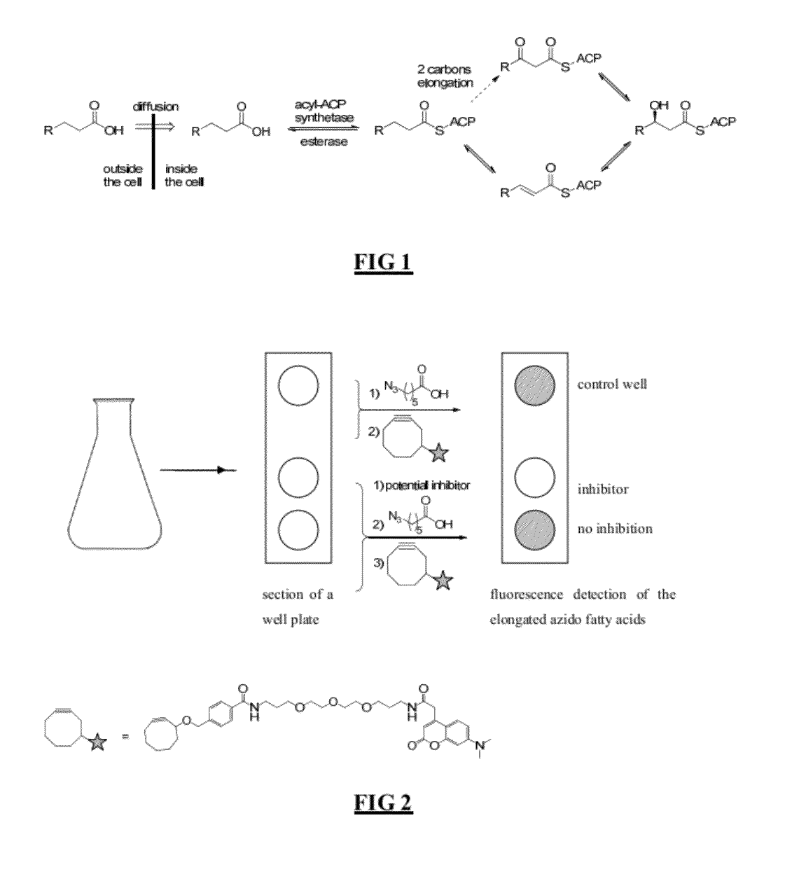

In some embodiments, elongation of a fatty acid occurs through elongation of an acyl chain by a cyclic repetition of four transformations that results in a two-carbon extension per cycle (FIG. 1). When adequate chain lengths are obtained (e.g. chain lengths typically ranging from 14 to 18 carbons), the fatty acids can be diverted from the elongation cycle and introduced into cell lipids, which can comprise thousands of different molecular species.

The term “cell” as used herein indicates the basic structural and functional unit of all known living organisms and it comprises eukaryotic and prokaryotic cells such as bacteria, archea, plat cells, fungi cells, protozoas, cells in multicellular organisms, also including cell lines and additional cells identifiable by a skilled person. Fatty acids are the basic components of many cellular lipids. Fatty acid elongation can require different enzymes in different cells, e.g. different bacteria and it is exemplary to have one multifunctional mega-enzyme for fatty acid elongation in mammals. However, the chemical transformations that characterize fatty acid elongation are typically very similar from one organism to another and are identifiable by a skilled person.

In several embodiments, methods and systems herein described are based on detection of fatty acid elongation of exogenous fatty acid precursors in a cellular environment. In particular, detection of changes in fatty acid elongation in a cellular environment according to methods and systems herein described allow in several embodiments identification of compounds capable of altering and in particular inhibiting fatty acid biosynthesis, identification of suitable concentrations of compounds capable of altering fatty acid biosynthesis, identification of cells capable of elongating exogenous fatty acids, identification of labeled fatty acid precursors capable of being elongated in a cell, and/or identification of conditions suitable for elongation of exogenous fatty acids in a cell.

In particular, in several embodiments herein described the methods comprise contacting an exogenous fatty acid precursor with a cell to allow incorporation of the exogenous precursor in the cells and allowing elongation of the precursor through a fatty acid elongation pathway occurring in the cell. In several embodiments, elongation of the fatty acid precursor can be monitored by way of a labeling the fatty acid precursor, as will be described herein in further detail. Thus, the ability to incorporate exogenous fatty acids into a cell and a subsequent detection of the incorporation, allows, under suitable conditions, an identification of compounds capable of altering fatty acid biosynthesis, identification of suitable concentrations of compounds capable of altering fatty acid biosynthesis, identification of cells capable of elongating exogenous fatty acids, identification of labeled fatty acid precursors capable of being elongated in a cell, and identification of conditions suitable for elongation of exogenous fatty acids in a cell. Reference is made to the Examples section and in particular to Example 1 wherein the ability to monitor the elongation through a labeled exemplary precursor is illustrated.

Suitable fatty acid precursors that can be used in methods and systems herein described comprise any compound that can be converted into a fatty acid through a suitable pathway and are identifiable by a skilled person. A suitable fatty acid precursor can typically be a fatty acid ranging from approximately 2 to 10 carbons such that it is short enough to allow elongation and to be incorporated prior to elongation. Suitable fatty acid precursors can also include compounds that can be converted to a substrate of a fatty acid elongation pathway such as esters and thioesters of fatty acids that can be hydrolyzed by esterase or amides of fatty acids that can hydrolyzed amide hydrolase as well as additional compounds identifiable to a skilled person. In several embodiments a precursor is cell permeable and water soluble. In particular, in several embodiments a suitable fatty acid precursor is cell permeable, water soluble, and is a substrate of a fatty acid elongation pathway. Fatty acid precursors which are cell permeable and water soluble are identifiable by a skilled person, for example, based on structural features of the fatty acid precursors. Methods are identifiable to a skilled person to specifically test whether a fatty acid precursor which is water soluble and/or cell permeable.

An exemplary test suitable to verify cell permeability is a Caco-2 permeability test, an assay that can be performed with a Caco-2 cell line which is derived from a human colon carcinoma. The Caco-2 cell line has characteristics which resemble intestinal epithelial cells, such characteristics comprising a forming of a polarized monolayer and well-defined brush borders on apical surfaces and intercellular junctions. A Caco-2 permeability test can be used to assess transport across the Caco-2 cell monolayer and thus can be used to access permeability. Accordingly, in some embodiments, a Caco-2 permeability test can be used to access cell permeability of a fatty acid precursor. A cell permeability or lack thereof of a fatty acid precursor to be tested (or already tested) for their ability to be elongated, can be used to distinguish a negative result originating from a lack of cell permeability from a negative result for a precursor which is able to enter the cell but is not able to be elongated by the cell.

An exemplary test to verify water solubility includes adding a predetermined amount of water (e.g. approximately 6 drops of water) to a test tube containing the precursor and shaking and/or stifling the tube until a homogeneous solution with water (if the precursor is water soluble), or a separate phase (if precursor is not water soluble) can be detected. Additional water, (e.g. up to 1 mL) can be added the precursor shows an ability to dissolve in water but does not not completely dissolve with the smaller amount. Additional parameter of the water can be checked (e.g. pH and/or saline concentrations) to determine whether the precursor is partially or completely soluble in water and whether the precursor has changed the pH or other conditions of the water as will be understood by a skilled person.

Suitability of a molecule to be a precursor can be verified in view of the pathway at issue based on the chemical properties of the precursor and the reactions involved as will be understood by a skilled person. In some embodiments the precursor can be the starting compound that is elongated through the fatty acid elongation pathway. In some embodiments, the precursor can be a compound that is incorporated in the fatty acid chain during elongation (e.g. in pathway directed to produce branched fatty acids).

Examples of precursors for the saturated straight chain fatty acid elongation pathway comprise, pyruvate, acetyl-coenzyme A, thioester, malonyl-coenzyme A, and thioesters.

Examples of precursors for unsaturated fatty acid elongation pathways include, but are not limited to, β-hydroxydecanoyl-acyl carrier protein, saturated fatty acid-CoA thioesters (including, but not limited to stearoyl-CoA).

Examples of precursors for the branched-chain fatty acid elongation pathways include, but are not limited to, branched amino acids (such as valine, leucine, and isoleucine), α-keto acids (such as α-ketoisovalerate, α-ketoisocaproic acid, and α-keto-β-methylvaleric acid) and their respective coenzyme A thioesters, branched carboxylic acids (such as isovaleric acid, isobutyric acid, and 2-methylbutyrate), and labeled versions of the aforementioned precursors.

In some embodiments, a fatty acid precursor is marked with a suitable label to produce a labeled fatty acid precursor which can be detected following elongation. The terms “label”, “labeled molecule” as used herein as a component of a complex or molecule refer to a molecule capable of detection, including but not limited to molecules emitting a labeling signal and molecules capable of binding with a compound emitting a labeling signal (e.g. through a functional group capable of reacting with a corresponding functional group on the compound emitting the signal). Exemplary molecules capable of direct detection comprise as radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion thereof which is capable of exhibiting detectable fluorescence. As a consequence, the wording “labeling signal” as used herein indicates a detectable signal that allows detection of the label, including but not limited to radioactivity, fluorescence, chemiluminescence, production of a compound in outcome of an enzymatic reaction and the like. In some embodiments the labeling signal is emitted directly from the label, in some embodiments the labeling signal is emitted from a compound attached to the label.

In methods and systems herein described, the label can be comprised in the precursor typically before exposing the precursor to the elongation pathway. In particular in embodiments wherein the precursor is labeled before elongation can occur, the label is selected so that it does not interfere with the chemical reactions of the elongation pathway (e.g. a radioactive label or azide functional group).

In some embodiments, the label is comprised in the precursor, emits a labeling signal and is capable of direct detection (e.g. radioactive carbon atoms). In some embodiments, the label is comprised in the precursor, is capable of binding to a compound emitting a signal (e.g. a probe comprising a dye) and is therefore capable of indirect detection. In some embodiments a label can be a functional group capable of reacting with a complementary bioorthogonal functional group on a labeled probe.

The term “bioorthogonal” as used herein indicates a functional group that is compatible in a biological system and, in particular, compatible with a cell. More particularly, a bioorthogonal functional group is relatively inert with respect to functional groups found in living systems, the functional groups found in living systems includes but not limited to alcohols, ammonium ions, disulfides, molecular oxygen, imidazolyl groups, phosphates, thiols, water, carboxylates, and bicarbonate.

For example, a bioorthogonal group can comprise a functional group which is not found in living systems, for example, an azide or an alkyne. However, bioorthogonal functional groups are not limited to those not found in living systems, for example bioorthogonal functional groups can comprise an aldehyde or ketone and additional functional groups identifiable by a skilled person.

For assays concerning a fatty acid elongation pathway, a selection of a bioorthogonal group on a fatty acid precursor suitable for elongation in a fatty acid elongation pathway in a cell can be performed such that the length of the labeled fatty acid precursor mimics the length of a corresponding natural fatty acid precursor which is capable of elongation. For example, an azide group can mimic an ethyl group. Accordingly in embodiments of the present disclosure, fatty acid precursors comprising an azide label comprise two less carbons in the chain than a corresponding natural fatty acid precursor. Thus, for other chemical reporters, the length of the fatty acid labeled fatty acid precursor can be selected based on the length and volume of a selected chemical reporter.

The term “bioorthogonal reaction” as used herein indicates a chemical reaction between a molecule comprising a label (e.g. a chemical reporter) and a probe molecule, the chemical reaction taking place at a rate which faster than a chemical reaction between a molecule in a cell and/or in vivo with either one of the molecule comprising the label and/or the probe molecule, to provide a selective reaction.

Examples of complimentary pairs of bioorthogonal functional groups include an azide and alkyne pair, an aldehyde or ketone and amine pair, an azide and phosphine pair, an alkene and terazine or tetrazole pair, however complimentary pairs of bioorthogonal functional groups are not limited to these examples. Examples of complimentary pairs of bioorthogonal functional groups and bioorthogonal reactions that can be used in embodiments of the present disclosure can be found in Bertozzi et al. (Bioorthogonal Chemistry: Fishing for Selectivity in a Sea of Functionality, Angew. Chem. Int. Ed. 2009, 48, 2-27), incorporated herein by reference in its entirety.

In some embodiments, a labeled precursor is provided in a form suitable for elongation. Methods for preparing the labeled precursors are identifiable by a skilled person upon reading of the present disclosure and include, for example, the syntheses in Hang et al. (J. Am. Chem. Soc. 2009, 131, 4967-4975), herein incorporated by reference in its entirety, and will not be further discussed in detail.

In embodiments of the disclosure, the precursor can be provided in a water soluble form, for example, as a salt or as a free acid in a mixture with a solubilizing agent such as a protein (e.g. BSA) or a detergent to favor water solubility. Salts of a precursor can be selected from non-toxic salts, in particular salts that are not toxic to the cell in which the labeled precursor is to be introduced, for example sodium, potassium, calcium, lithium, magnesium, ammonium, and other salts identifiable by a skilled person upon reading the present disclosure. Detergents suitable to be used in connection with a suitable precursor in methods and systems herein described can be selected from non-toxic detergents, in particular detergents that are not toxic to the cell in a concentration in which the detergent along with the labeled precursor is to be introduced. Detergents of the disclosure include, but are not limited to anionic, cationic, zwitterionic, and/or neutral detergents.

In some embodiments, methods and systems herein described are directed to identification of a compound able to affect a fatty acid elongation pathway.

The term “alter” or “affect” as used herein with reference to a fatty acid elongation pathway and in particular to fatty acid biosynthesis relates to the ability to act on; produce an effect or change in the biosynthesis. Compounds able to affect a pathway typically comprise inhibitors and enhancers of the pathway. The term “inhibitor” as used herein with reference to a fatty acid elongation pathway and in particular to fatty acid biosynthesis indicates a compound capable of interfering with a fatty acid elongation pathway in such a way that is associated to a decrease in concentration of a detectable product of the pathway compared to the concentration of a same product in the cell a system under a same set of conditions without the inhibitor (e.g. a control), wherein the detection can be performed through techniques identifiable by a skilled person. Suitable inhibitors comprise, compounds able causing decrease in detectable fatty acid produced by elongation of fatty acid precursors present in the cell. In particular, the decrease in the elongation of fatty acids can lead to a lower concentration of fatty acids being incorporated into the cell membrane compared to the control.

The term “enhancer” as used herein with reference to a fatty acid elongation pathway and in particular to fatty acid biosynthesis indicates a compound capable of interfering with a fatty acid elongation pathway in such a way that is associated to an increase in concentration of a detectable product of the pathway compared to the concentration of a same product in the cell a system under a same set of conditions without the inhibitor (e.g. a control), wherein the detection can be performed through techniques identifiable by a skilled person. Suitable enhancer comprise, compounds able causing increase in detectable fatty acid produced by elongation of fatty acid precursors present in the cell elongation of fatty acid precursors present in the cell a system under a same set of conditions without the enhancer (e.g. a control). In particular, the increase in the elongation of fatty acids can lead to a higher concentration of fatty acids being incorporated into the cell membrane compared to the control.

In several embodiments, candidate compounds can be contacted with a cell to verify the ability to interfere with a fatty acid elongation pathway. Candidate compounds to be tested for ability to alter a fatty acid elongation pathway can be any compound which is cell permeable. As previously mentioned in connection with testing cell permeability of a fatty acid precursor, a Caco-2 permeability test can be performed if it is desired to determine cell permeability of a candidate compound. Cell permeability of compounds which are tested for their ability to interfere with a fatty acid elongation pathway can be used to distinguish a negative result originating from a lack of cell permeability from a negative result for a compound which can enter the cell but in not capable of interfering with a fatty acid elongation pathway.

In embodiments directed to verify the ability of a candidate compound to affect the pathways, the contacting is performed for a time and under condition to allow interaction of the compound with the elongation pathway at issue in the cell as will be identifiable by a skilled person.

In some embodiments for example, a candidate compound can be contacted with a culture of cells grown in a defined medium which can first be loaded and supplemented with a labeled precursor, such as a short chain azido fatty acid (see Examples section). In some embodiments, the candidate compound and the labeled precursor can be contacted simultaneously. In some embodiments the candidate compounds can be contacted before contacting the cell with the candidate precursor. In some embodiments, the precursor is not significantly incorporated into cellular lipids prior to contacting of the candidate compound. In some of those embodiments, the compound and the precursor can be contacted in a cell-based high throughput screening assay performed with a culture of cells grown in a defined medium n a well plate. In some embodiments, the precursor is not significantly incorporated into cellular lipids prior to elongation. The contacting of the candidate compound and of the precursor is typically performed for a time and under condition to allow incorporation of those compounds in the cell based on the specific water solubility, cell permeability and reaction conditions identifiable by a skilled person.

In some embodiments, cells to be used in the assay are capable of elongating the precursor through their fatty acid elongation machinery (see FIG. 1). Suitable cells comprise for example Vibrio harveyi strain B392 which is capable of elongating N₃(CH₂)₅CO₂H as well as other short chain fatty acid precursors added to the culture medium. In some embodiments, excess short chain fatty acid precursor in the medium can be removed away from the cells. In particular, excess short chain fatty acid precursors can be washed away with aqueous solutions, and more particularly with a saline solution. In some embodiments, wherein the label does not emit the labeling signal and instead binds to a compound emitting the labeling signal (e.g. a dye) the compound emitting the labeling signal can be remove before detection.

In some embodiments, the contacting is followed by detecting the labeling signal from the labeled precursor following elongation. The terms “detect” or “detection” as used herein indicates the determination of the existence, presence or fact of a target (e.g. elongated fatty acid) in a limited portion of space, including but not limited to a sample, a cell, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

In some embodiments, detecting the labeling signal can be performed following removal of the labeled precursor. In particular in some of those embodiments, following removing of the labeled precursor remaining in the medium (e.g. a washing out of the excess short chain fatty acid), cells that have incorporated the precursor into their lipids can be tested

In some embodiments, wherein the precursor is a labeled precursor, the detecting can be performed following the contacting, possibly after removal of the labeled precursor. In some embodiments, wherein the precursor is configured to include a bioorthogonal functional group able to bind a label, the detecting is performed by contacting the precursor with a probe comprising the label to allow binding of the functional group in the precursor with the functional group in the probe or label and then detecting the label

In particular in exemplary embodiments wherein the label is a fluorescent molecule, detection of fatty acid elongation can be performed after treatment with a dye that reacts specifically with the label at issue, or after washing away the excess of unreacted dye, for example with a saline solution.

In several embodiments, the dye can comprise a fluorophore or a chromophore within a probe further comprising functional groups bioorthogonal with the functional group in the precursor. Suitable dyes that can react with an azide group comprise a cyclooctyne or alkyne-dye conjugate that reacts specifically with organic azides (Ref. 5 and 6)

In particular if the functional group is an azide functional group a cyclooctyne dye or other suitable dye that reacts specifically with the azide functional group (see Examples section) can be used. In some embodiments, the azido functional group or other functional group into their lipids can be detected after treatment with a cyclooctyne dye or other suitable dye through other types of luminescence.

Exemplary methods and instruments for detecting a luminescent signal comprise flow cytometry and plate readers, and further detection methods such as fluorescence microscopy, fluorimeter, fluorescence activating cell sorting (FACS), fluorescence resonance energy transfer (FRET) and additional methods identifiable by a skilled person. In particular, additional techniques can be found in Bertozzi et al. (Bioorthogonal Chemistry: Fishing for Selectivity in a Sea of Functionality, Angew. Chem. Int. Ed. 2009, 48, 2-27), incorporated herein by reference in its entirety and further are identifiable by a skilled person upon reading of the present disclosure and will not be further discussed in detail.

In embodiments, where a dye is used cells treated with an inhibitor of fatty acid elongation, the elongated azido fatty acid will not incorporate the azido group or other label and will therefore remain ‘dark’ after treatment with the dye. For example, in embodiments wherein cells are contacted with inhibitors the fluorescence measured after treatment with an inhibitor can be significantly decreased compared to the same culture untreated (see Examples section).

In embodiments, where a dye is used cells treated with an enhancer of fatty acid elongation prior to addition of the azido fatty acid will incorporate the azido group or other label and will therefore produce a more intense detection signal than a control after treatment with the dye. For example, in embodiments wherein cells are contacted with enhancer the fluorescence measured after treatment with the enhancer can be significantly increased compared to the same culture untreated. (see Examples section)

Additional labels and related detection techniques can be used instead or in addition to luminescence detection as will be understood by a skilled person.

In some embodiments, detection of the elongated fatty acids is a qualitative detection. In those embodiments, the methods and systems are directed to identify whether elongation of the fatty acid has occurred through a qualitative detection of the signal. In some of those embodiments, the qualitative detection of the signal can be used to screen and identify inhibitor and enhancer of the pathway (see for example cerulenin which can provide a seven fold or more decrease in fluorescence compared to an untreated cell, see Example 1, see also Example 8).

In some embodiments, detection of the elongated fatty acid is a quantitative detection. In those embodiments, detection of amounts of elongated fatty acid is performed through quantitative detection of the signal. In some of those embodiments, the quantitative detection of the elongated fatty acid is indicative of a degree of inhibition, enhancement or other forms of interference of a compound with the elongation pathway at issue. In particular, detection of a different increase or decrease in fluorescence or other signal can also be informative of the enhancing or inhibiting ability of the compound at issue (see for example cerulenin which can provide a seven fold or more decrease in fluorescence compared to an untreated cell, see Example 1, and see also Examples 8 and 9).

In some embodiments, detection of the labels can be performed on the cell (e.g. on the membrane of the cells incorporating the elongated fatty acids, see e.g. the Examples section). In some embodiments, detection of the fatty acids can be performed following rupturing of the cell and/or isolation of the elongated fatty acids from the cell. Additionally, in some embodiments, the elongated labeled fatty acid can react with protein, for example, by esterification. Thus, in some of those embodiments, a protein or a mixture of proteins capable of reacting with a fatty acid can be detected. A skilled person will be able to identify techniques and procedures to perform detection of the elongated labeled fatty acids maintaining the cell intact or following treatment of the cell directed to provide cell extracts and/or isolate those fatty acids derivatives.

In some embodiments, identification of a compound suitable to affect fatty acid elongation pathway can be performed in a cell-based high throughput screening assay. In particular, the assay can be performed for the identification of fatty acid biosynthesis inhibitors. In some of those embodiments, the assay is specifically designed to identify potential drug candidates that inhibit one or more of the enzymes required in the metabolic pathway of fatty acid elongation.

In some embodiments, identification of a compound that can affect and in particular inhibit fatty acid elongation can be used in antibiotic discovery, in particular in embodiments where the cells are validated cellular targets for antibiotic development for which different enzymes are targeted by currently used antibiotics. Inhibitors of fatty acid elongation pathway in those cases are suitable to be used as antibiotics alone or in combination with other antibiotics as would be understood by a skilled person. In particular, in some of those embodiments, methods and systems herein described allow antibiotic discovery at a rate at which potential drug candidate can be tested higher than some approaches currently used. In some of those embodiments, methods and systems herein described allow performing screening assays without requiring isolation of one or more targeted enzyme prior to in vitro testing. In some embodiments, methods and systems herein described provide information about the behavior of the drug candidate in a cellular environment or about its cellular permeability.

In some embodiments, identification of a compound that can affect and in particular inhibit fatty acid elongation can be used in connection with identifying compounds with apoptotic activity. In this connection, it has been shown that inhibitors of human fatty acid biosynthesis induce selective apoptosis cells such as in cancer cells (Ref. 3). Thus, in some embodiments, identifying a compound capable of altering a fatty acid elongation pathway can provide candidate compounds for a further screening of compounds having apoptotic activity.

In some embodiments, the method comprises identifying a compound capable of inhibiting a fatty acid elongation pathway according to methods herein described. The method further comprises contacting the identified compound with the cell for a time and under conditions to allow interference of the compound with the fatty acid elongation pathway; and detecting viability of the cell following the contacting

In particular detecting viability of the cell can be performed by performing a test on the compound identified as capable of inhibiting the fatty acid elongation pathway to confirm apoptotic activity.

In some of those embodiments, further tests to be performed on a compound identified as capable of inhibiting the fatty acid elongation pathway to confirm apoptotic activity include various apoptosis assays, for example apoptosis assays using nucleic acid stains, apoptosis assay that detect DNA strand breaks, apoptosis assays that detect membrane asymmetry, apoptosis assays based on protease activity, apoptosis assays using mitochondrial stains, and live/dead assays such as those offered by INVITROGEN™ or other assays as described in described in PROMEGA's “Protocols and Applications Guide”, Cell Viability, 4, rev. 3/11 and in “Total Cytotoxicity & Apoptosis Detection Kit, A direct assay to accurately quantify cytotoxicity including cells in early apoptosis” by Immunochemistry Technologies LLC, both of which are herein incorporated by reference in their entirety. Other tests for apoptotic activity are identifiable by a skilled person upon reading the present disclosure.

In some embodiments herein described, methods can be directed to identification of a compound capable of altering the fatty acid elongation pathway that is capable of enhancing fatty acid elongation. Compounds capable of enhancing fatty acid elongation can be used in connection with various applications, including for example, production of biofuels, and in particular biodiesel.

The term “biofuel” as used herein indicate a fuel derived from biological carbon fixation (e.g. from a reduction of CO₂ to functionalized or unfunctionalized hydrocarbons by living organisms). For example, biodiesel is a type of biofuel comprised of long-chain fatty acids (e.g. fatty acids comprising approximately 14-30 carbon atoms).

In embodiments of the disclosure, in which a concentration of a candidate compound potentially or known to be capable of affecting fatty acid elongation has been identified as inhibiting growth of the cell where the assay is to be performed, the assays can be performed using a concentration of a candidate compound which is less than the growth inhibitory concentration of the compound to minimize growth inhibition of a cell since cell growth allows for fatty acid elongation.

In some embodiments where minimization of false positives is desired, the assay can be performed with concurrent monitoring of expression of a fluorescent protein, or any other viability marker. For example the cell viability assays described in PROMEGA's “Protocols and Applications Guide”, Cell Viability, 4, rev. 3/11, herein incorporated by reference in its entirety, to provide information about cell protein synthesis and viability and by additional methods identifiable by a skilled person.

In further embodiments of the disclosure, methods for identifying cells capable of elongating short chain azido fatty acids through their fatty acid elongation machinery. For example, a cell-based high throughput screening assay can be performed with one or more cultures of cells grown in a defined medium which can first be loaded in a well plate and then supplemented with a labeled precursor such as a short chain azido fatty acid (see Examples section) or other labeled fatty acid precursor identifiable by a skilled person. Excess short chain fatty acid remaining in the medium can be removed and the labeled precursor detected (e.g. in case where the precursor is a short chain azido fatty acid followed by a treatment with a cyclooctyne dye or other suitable dye that reacts specifically with the azide functional group (see Examples section). Cells that have incorporated the azido functional group or other label into their lipids can become fluorescent after treatment.

Thus, cells capable of elongating fatty acids (e.g. cells comprising the enzymes for fatty acid elongation) can incorporate the azido functional group or other label into their lipids and can thus be detected after treatment with a cyclooctyne dye or other suitable dye. Cells which are not capable of elongating fatty acids (e.g. cells which do not comprise the enzymes for fatty acid elongation) will not incorporate the azido group or other label into their lipids and will therefore remain ‘dark’ after treatment with the dye.

Cells identified by methods and systems herein described in some embodiments, can in turn be used in an assay to identify compounds capable of altering a fatty acid elongation pathway.

In some embodiments, methods and systems to detect fatty acid elongation pathway can be applied to identify a concentration of a compound effective for altering a fatty acid elongation pathway. For example, once a compound is identified as being capable of altering a fatty acid elongation pathway, either by methods of the disclosure, or based on a known property of a compound, embodiments of the disclosure also provide a method to determine a concentration or range of concentrations suitable for altering a fatty acid elongation pathway.

In some embodiments, the method is performed by contacting the compound with the cell a concentration with a labeled fatty acid precursor and a cell to allow interference of the compound with the fatty acid elongation pathway and then detecting the signal to determine a concentration that is effective in altering the fatty acid elongation pathway.

In those embodiments, the method can comprise performing a cell-based high throughput screening assay using various concentrations of a selected compound capable of altering a fatty acid elongation pathway. In particular, in some of those embodiments the method can further comprise contacting the one or more compounds capable of altering the fatty acid elongation pathway each at one or more second concentrations, and a labeled fatty acid precursor with the cell comprising the enzymes required in the fatty acid elongation pathway, the contacting performed for a time and under condition to allow one or more second elongations of the labeled fatty acid precursor through the fatty acid elongation pathway and to allow interference of the compound at each of the one or more second concentrations with the fatty acid elongation pathway. The method also comprises detecting one or more second labeling signals associated with the labeled fatty acid precursor following the one or more second elongations each labeling signal associated with each one or more second concentrations. The method further comprises comparing the first labeling signal with the one or more second labeling signals to determine one or more effective concentrations in altering the fatty acid elongation pathway.

The wording “associated with” or “associated to” as used herein with reference to two items indicates a relation between the two items such that the occurrence of a first item is accompanied by the occurrence of the second item, which includes but is not limited to a cause-effect relation relation.

In some embodiments, the methods can be performed with cell cultures grown in a defined medium loaded in a well plate and then supplemented with a short chain azido fatty acid or other labeled fatty acid precursor herein described. Excess short chain fatty acid remaining in the medium can be removed and the cells treated with a cyclooctyne dye or other suitable dye that reacts specifically with the azide functional group.

Cells that that have incorporated the azido functional group or other label into their lipids can become fluorescent or otherwise detectable after treatment. Thus, if a compound enhances fatty acid elongation, a higher concentration of the compound can lead to a higher intensity detection signal (compared to lower concentration of the same compound). If a compound is an inhibitor of fatty acid elongation, a higher concentration of the compound can lead to a lower intensity signal (compared to lower concentration of the same compound). Thus intensity of the detection signal can be used to compare different concentrations tested.

Determination of an effective concentration of a compound for altering a fatty acid elongation pathway can be used in connection with determining a minimum effective concentration, for example, to minimize side effects and/or toxicity of a compound to be used in connection with a treatment of a disease or to look for a most effective concentration of a compound, depending on the application.

In some embodiments, a compound identified as altering a fatty acid elongation pathway (e.g. according to methods herein described directed to identify such a compound) can be further characterized to identify a mechanism of action by which the compound alters the fatty acid elongation pathway. A mechanism of action can be identified, for example, by identifying the ability of the compound inhibition of one or more individual enzymes involved in the fatty acid elongation pathway. In vitro inhibition assay are available for most enzymes in the fatty acid elongation pathway. See, for example Rock et al. (Biochimica et Biophysica Acta 1302 (1996) 1-16), herein incorporated by reference in its entirety.

Further embodiments of the disclosure provide a method to determine one or more parameters and in particular, one or more value and/or range of values for the one or more parameters under which a cell is capable of elongating an exogenous fatty acid. For example, the value and/or range of values of a parameter can be a concentration and/or range of concentrations of a labeled fatty acid precursor used in an assay; a temperature and/or range of temperatures at which the assay is performed; and other parameters identifiable by a skilled person. In those embodiments, the contacting is performed for a time and under condition associated with the one or more parameter and in particular to the related value at issue. Detection of the signal following elongation performed for each of the value tested allows determination of the corresponding effect on the fatty acid elongation.

For example, in some of those embodiments methods can comprise performing a cell-based high throughput screening assay using various concentrations of a labeled fatty acid precursor with cell cultures comprising cells capable of elongating fatty acids and grown in a defined medium loaded in a well plate. Excess short chain fatty acid remaining in the medium can be removed and the label detected according to techniques and procedures described herein. For example, in embodiments wherein the cells that have incorporated the azido functional group or other label into their lipids can become fluorescent after treatment. A desired concentration or range concentrations of the labeled fatty acid precursor to be used in an assay can be selected based on a desired intensity of the signal.

The desired intensity of the signal can be selected in connection with a desired screening objective. For example, if the concentration of labeled fatty acid precursor is being determined for use in an assay for screening for fatty acid inhibitors, a stronger signal can be desired so that a positive result for an inhibitor (e.g. a diminished signal) can be more apparent. If the concentration of a labeled fatty acid precursor is being determined for use in an assay screening for enhancers of fatty acid elongation, a weaker signal can be desired so that a positive result for an inhibitor (e.g. an enhanced signal) can be more apparent.

Additional parameters that can be tested to identify a value or range of values able to affect a fatty acid elongation pathway comprise, media composition, temperature, number of carbon in the fatty acid precursor, introduction time point for a compound able to affect fatty acid elongation pathway, and/or of other compound, and additional parameters identifiable by a skilled person

As described herein, at least two of the labeled fatty acid precursor, cells and reagents for detection of the label herein described can be provided as a part of systems to perform any assay, including any of the assays described herein. The systems can be provided in the form of kits of parts. In a kit of parts, the labeled fatty acid precursor, and other reagents to perform the assay can be comprised in the kit independently. The primers can be included in one or more compositions, and each labeled fatty acid precursor can be in a composition together with a suitable vehicle.

Additional components can include labeled molecules and in particular, labeled polynucleotides, labeled antibodies, labels, microfluidic chip, reference standards, and additional components identifiable by a skilled person upon reading of the present disclosure.

In particular, the components of the kit can be provided, with suitable instructions and other necessary reagents, in order to perform the methods here described. The kit will normally contain the compositions in separate containers. Instructions, for example written or audio instructions, on paper or electronic support such as tapes or CD-ROMs, for carrying out the assay, will usually be included in the kit. The kit can also contain, depending on the particular method used, other packaged reagents and materials (i.e. wash buffers and the like).

Further advantages and characteristics of the present disclosure will become more apparent hereinafter from the following detailed disclosure by way of illustration only with reference to an experimental section.

### EXAMPLES

The methods and systems herein disclosed are further illustrated in the following examples, which are provided by way of illustration and are not intended to be limiting.

In particular, the following examples illustrate exemplary assay for identifying inhibitors of fatty acid elongation pathway and related methods and systems. In particular, the following examples illustrate elongation of a short chain fatty acid precursor comprising an azide group in Vibrio harveyi strain B392 cells and related detection using a cyclooctyne-coumarin conjugate dye. In particular, in the examples below elongation of fatty acid is performed in connection with methods and systems directed to identify inhibitors, related concentrations, suitable cells and reaction conditions related to the pathway. A person skilled in the art will appreciate the applicability and the necessary modifications to adapt the features described in detail in the present section, to additional precursors, labels, cells and in particular cellular microorganisms, solutions, methods and systems according to embodiments of the present disclosure.

### Example 1

### Detection of Fatty Acid Elongation Following Contacting with Known Fatty Acid Biosynthesis Inhibitor Cerulenin

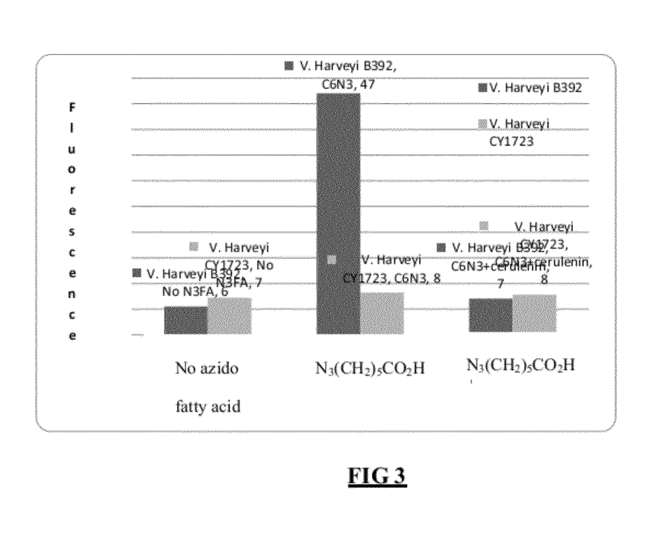

Fatty acid elongation has been detected in Vibrio harveyi strain B392 using experimental settings schematically illustrated in FIG. 2.

Vibrio harveyi strain B392 (Ref. 7) is capable of elongating and incorporating exogenous short to medium chain azido fatty acids added to the culture medium (See Example 3), and in particular, short chain azide fatty acids that are 4 to 8 carbons long as well as medium chain azido fatty acids that are 10 carbons or more. In particular, N₃(CH₂)₅CO₂H(C₆N₃) shows the highest level of incorporation in Vibrio harveyi B392, and was thus selected for use in the assay. In the example described here, the fluorescence measured after treatment with a cyclooctyne-coumarin conjugate was elevated compared to the same culture treated with cerulenin, a known fatty acid elongation inhibitor (Ref. 8). V. harveyi B392 that were not treated with C₆N₃ also showed low fluorescence. V. harveyi strain CY1723 (Ref. 7) is a mutant that has limited exogenous fatty acid elongation capability even without antibiotic treatment and was considered to be used as a control.

In particular, the cells were grown in 5 mL of synthetic media (Ref 9), and at OD 0.2, cerulenin was introduced to appropriate samples for a final treatment concentration of 10 μg/mL (sub-bacteriostatic). Then, at OD 0.3, C₆N₃ was introduced to appropriate samples for a final treatment concentration of 5 mM. After allowing the cells to grow in presence of C₆N₃ for 5 hours, the cells were spun down at 5000 g for 10 minutes, and the supernatant was removed. Non-incorporated C₆N₃ was washed from cells three times by re-suspending the cell pellet in 10 mL of 0.9% NaCl, spinning the cells down at 5000 g for 10 minutes, and removing the supernatant. This washing procedure was repeated two more time. The cells were re-suspended in 1 mL of 50 μM cyclooctyne-coumarin conjugate in 0.9% NaCl and incubated at 37° C. for 30 minutes. Cells were collected and washed three more times with 10 mL of 0.9% NaCl to remove non-reacted cyclooctyne-coumarin conjugate, and a fraction of the cells re-suspended in 2 mL of 0.9% NaCl were analyzed by plate reader (excitation 380 nm, detection 500 nm) or flow cytometry (excitation 407 nm, detection 450/40 bandpass filter).

Results of these experiments are illustrated in FIG. 3. In particular, the results of FIG. 3, indicate that the fluorescence measured after treatment with a cyclooctyne-coumarin conjugate was elevated about seven-fold compared to the same culture treated with cerulenin, a known fatty acid elongation inhibitor (FIG. 3). V. harveyi B392 that were not treated with N₃(CH₂)₅CO₂H also showed low fluorescence. V. harveyi strain CY1723 is a mutant that has limited exogenous fatty acid elongation capability and was used as a control. Results of these experiments are illustrated

It is noted that in these experiments, B392 is used in well plates for simpler loading (FIG. 2), however B392 and CY can be used interchangeably as a control (see Example 2 below).

### Example 2

### Identification of Cells Suitable to be Used with Fluorescent Label

In order to find the most appropriate negative control (e.g. the one that gives the lowest Fluorescence/OD (or the highest ratio)) for the determination of fluorescence due to background labeling, B392 and CY1723 were subject to various conditions involving the presence of C₆N₃ and cerulenin (Ref 8), a fatty acid biosynthesis inhibitor.

After cells were allowed to grow for 5 hours, extracellular C₆N₃ was washed away with 0.9% NaCl, and cells were treated with 50 μM cyclooctyne-coumarin conjugate for 30 minutes at 37° C. Non-reacted cyclooctyne-coumarin conjugate was washed away with 0.9% NaCl, and cells were re-suspended in 0.9% NaCl to be analyzed by plate reader (TABLE 1).

In particular, the cells were grown in 5 mL of synthetic media (Ref 9), and at OD 0.2, cerulenin was introduced to appropriate samples for a final treatment concentration of 10 μg/mL (sub-bacteriostatic). Then, at OD 0.3, C₆N₃ was introduced to appropriate samples for a final treatment concentration of 5 mM. After allowing the cells to grow in presence of C₆N₃ for 5 hours, the cells were spun down at 5000 g for 10 minutes, and the supernatant was removed. Non-incorporated C₆N₃ was washed from cells three times by re-suspending the cell pellet in 10 mL of 0.9% NaCl, spinning the cells down at 5000 g for 10 minutes, and removing the supernatant. The cells were re-suspended in 1 mL of 50 μM cyclooctyne-coumarin conjugate in 0.9% NaCl and incubated at 37° C. for 30 minutes. Cells were collected and washed three more times with 10 mL of 0.9% NaCl to remove non-reacted cyclooctyne-coumarin conjugate, and cells re-suspended in 2 mL of 0.9% NaCl were analyzed by plate reader (excitation 380 nm, detection 500 nm) or flow cytometry (excitation 407 nm, detection 450/40 bandpass filter).

TABLE 1

| Entry | Strain | C₆N₃ | Cerulenin | Fluorescence/OD | Ratio^{a} |
|---|---|---|---|---|---|
| 1 | B392 | + | − | 528217 | 1 |
| 2 | B392 | + | + | 45609 | 11.58 |
| 3 | B392 | − | − | 90733 | 12.42 |
| 4 | CY1723 | + | − | 162321 | 3.25 |
| 5 | CY1723 | + | + | 44069 | 11.99 |

In TABLE 1 above, the V. harveyi strain used is indicated in the first column, whether C₆N₃ and/or Cerulenin were present (+ present, − not present) is indicated in the second and third columns, and the fluorescence/OD measured by the plate reader is indicated in the fourth column. The fifth column is the fluorescence/OD of the B392 with only C₆N₃ (Entry 1) divided by the fluorescence/OD of the given sample. Thus, samples that were less fluorescent than the positive control sample (Entry 1) would have measured ratios greater than unity.

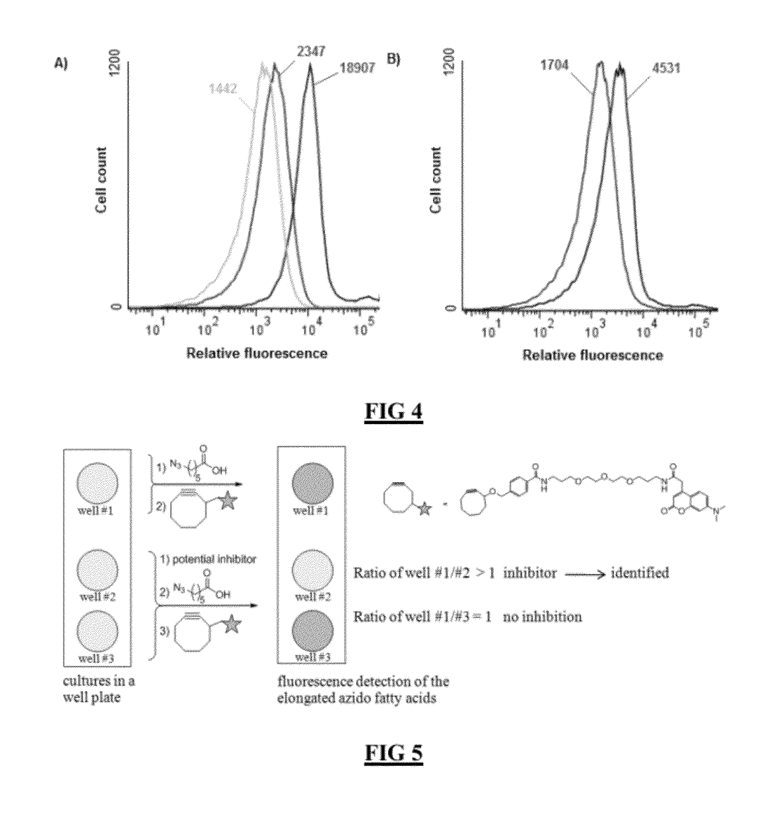

In addition, the samples were analyzed by flow cytometry (FIG. 4)

TABLE 2

| Entry | Strain | C₆N₃ | Cerulenin | Mean fluorescence | Ratio^{a} |
|---|---|---|---|---|---|
| 1 | B392 | + | − | 18907 | 1 |
| 2 | B392 | + | + | 2347 | 8.06 |
| 3 | B392 | − | − | 1442 | 13.11 |
| 4 | CY1723 | + | − | 4531 | 4.17 |
| 5 | CY1723 | + | + | 1704 | 11.09 |

In TABLE 2 above, the V. harveyi strain used is indicated in the first column, whether C₆N₃ and/or Cerulenin was present (+ present, − not present) is indicated in the second and third columns, and the mean fluorescence of the flow cytometry fluorescence histogram (FIG. 4) for a given sample is in the fourth column. The fifth column is the mean fluorescence of the B392 with only C₆N₃ (Entry 1) divided by the mean fluorescence of the given sample. Thus, samples that were less fluorescent than the positive control sample (Entry 1) would have measured ratios greater than unity.

The results in TABLES 1 and 2 above show that B392 cells not treated with C₆N₃ or cerulenin had the lowest fluorescence (Entry 3 of both tables). However, B392 and CY1723 cells treated with C₆N₃ and cerulenin had only slightly higher fluorescence (Entries 2 and 5 of both tables), which showed that cerulenin inhibited fatty acid biosynthesis efficiently and, therefore, interfered with azide incorporation in lipids.

### Example 3

### High-Throughput Assay to Identify Fatty Acid Biosynthesis Inhibitors Among Other Noninhibitors

In this example, a high-throughput cell-based antibiotic screen, which would identify fatty acid biosynthesis inhibitors, was implemented. B392 cells in a 96-well plate were treated with various concentrations of commercially available fatty acid biosynthesis inhibitors, including cerulenin (Ref. 8), bischloroanthrabenzoxocinone (Ref. 10), thiolactomycin (Ref. 11), and platensimycin (Ref. 2) as well as a number of arbitrarily chosen drugs (listed in TABLE 3).

TABLE 3

| Well | CAS | Name | MW |
|---|---|---|---|
| A2-A3 | 17397-89-6 | Cerulenin* | 223.3 |
| A5-A6 | 53847-30-6 | 2-Arachidonoylglycerol* | 378.5 |
| A7-A9 | 89464-63-1 | Dimethyloxaloylglycine | 175.1 |
| A11-A12 | 3102-57-6 | C2 Ceramide* | 341.5 |
| B2-B3 | 74772-77-3 | Ciglitazone* | 333.4 |
| B5-B6 | 55028-72-3 | Cloprostenol* | 424.9 |
| B8-B9 | 14152-28-4 | Prostaglandin A₁* | 336.5 |
| B11-B12 | 123-78-4 | Sphingosine* | 299.5 |
| C1-C3 | 6108-05-0 | Lidocaine•HCl•H₂O | 288.8 |
| C4-C6 | 91-40-7 | N-Phenylanthranilic acid | 213.2 |
| C7-C9 | 614-39-1 | Procainamide | 235.3 |
| C11-C12 | 50-02-2 | Dexamethasone* | 392.5 |
| D1-D3 | 53-86-1 | Indomethacin | 357.8 |
| D4-D6 | 22373-78-0 | Monensin | 692.9 |
| D7-D9 | 36322-90-4 | Piroxicam | 331.3 |
| D11-D12 | 1397-94-0 | Antimycin A* | 548.6 |
| E2-E3 | 1404-19-9 | Oligomycin* | 791.0 |
| E4-E6 | 6119-47-7 | Quinine•HCl•2H₂O | 396.9 |
| E7-E9 | 3544-24-9 | 3-aminobenzamide | 136.2 |
| E10-E12 | 57-41-0 | Phenytoin | 252.3 |
| F1-F3 | 665-66-7 | Amantadine•HCl | 187.7 |
| F4-F6 | 2016-88-8 | Amiloride•HCl | 266.1 |
| F8-F9 | 22862-76-6 | Anisomycin* | 265.3 |
| F10-F12 | 7689-03-4 | Camptothecin | 348.4 |
| G2-G3 | 58-58-2 | Puromycin•2HCl* | 530.4 |
| G4-G6 | 3380-34-5 | Triclosan | 289.5 |
| G7-G9 | 54-85-3 | Isoniazid | 137.1 |
| G12 | 866022-28-8 | Bischloroanthrabenzoxocinone | 543.4 |
| H3 | 82079-32-1 | Thiolactomycin | 210.3 |
| H6 | 835876-32-9 | Platensimycin | 441.5 |

In TABLE 3 above, rows of the 96-well plate are indicated by a letter and the columns are indicated by a number. Antibiotics labeled with a *, the wells contain 200× stocks of 10 mM and 1 mM of antibiotic in descending order. For bischloroanthrabenzoxocinone, thiolactomycin, and platensimycin, the wells only contain 200× stocks of 1 mM of antibiotic. For other antibiotics, the wells contain 200× stocks of 100 mM, 10 mM, and 1 mM of antibiotic in descending order.

B392 cells given no drug treatment served as the control. Cells were then fed with C₆N₃ and allowed to grow overnight. Extracellular C₆N₃ was washed away with 0.9% NaCl three times, and cellular lipids were click-reacted with 50 μM cyclooctyne-coumarin conjugate for 30 minutes at 37° C. Non-reacted cyclooctyne-coumarin conjugate was washed away with 0.9% NaCl three times, and cells were re-suspended in 0.9% NaCl to be analyzed by plate reader. Any cells grown in cultures containing fatty acid biosynthesis inhibitors would have significantly lower fluorescence in comparison to non-treated cells due to decreased C₆N₃ incorporation. Meanwhile, non-fatty acid biosynthesis inhibitors would have no significant effect on C₆N₃ incorporation and the fluorescence of cells. It should be noted that the same method described here in Example 3 can be used to identify compounds which enhance fatty acid biosynthesis. Any cells grown in cultures containing fatty acid biosynthesis enhancers would have significantly higher fluorescence in comparison to non-treated cells due to increased C₆N₃ incorporation.

In particular, B392 cells were grown in 150 mL cultures of synthetic media (Ref. 9) until OD 0.3 was reached. Cells were transferred (1 mL of the culture loaded into each well) to a 2 mL 96-well plate via a multi-pipette robot (this robot was used for most liquid transfers mentioned in this example). To each well, 5 μL of antibiotic from a 200× stock antibiotic plate in DMSO (TABLE 3) was introduced to create final treatment concentrations of 500 μM, 50 μM, and 5 μM. After 30 minutes, 10 μL of 500 mM C₆N₃ in DMSO was introduced to each well for a final treatment concentration of 5 mM. Cells were grown overnight, then spun down at 2000 g for 10 minutes. Supernatant was removed, and then non-incorporated C₆N₃ was washed from cells three times by re-suspending the cells in 1 mL of 0.9% NaCl, spinning the cells down at 2000 g for 10 minutes, and removing the supernatant. The cells were re-suspended in 200 μL of 50 μM cyclooctyne-coumarin conjugate in 0.9% NaCl and incubated at 37° C. for 30 minutes. Cells were collected and washed three more times with 1 mL of 0.9% NaCl to remove non-reacted cyclooctyne-coumarin conjugate, and cells re-suspended in 400 μL of 0.9% NaCl were analyzed by plate reader for fluorescence (excitation 380 nm, detection 500 nm) and cell density.

When considering the fluorescence ratio (see explanation of TABLES 1 and 2 in Example 1) between non-treated cells and drug treated cells, cells treated with fatty acid biosynthesis inhibitors would have a fluorescence ratio much greater than 1, while cells treated with non-fatty acid biosynthesis inhibitors would have a fluorescence ratio of approximately 1 (FIG. 5).

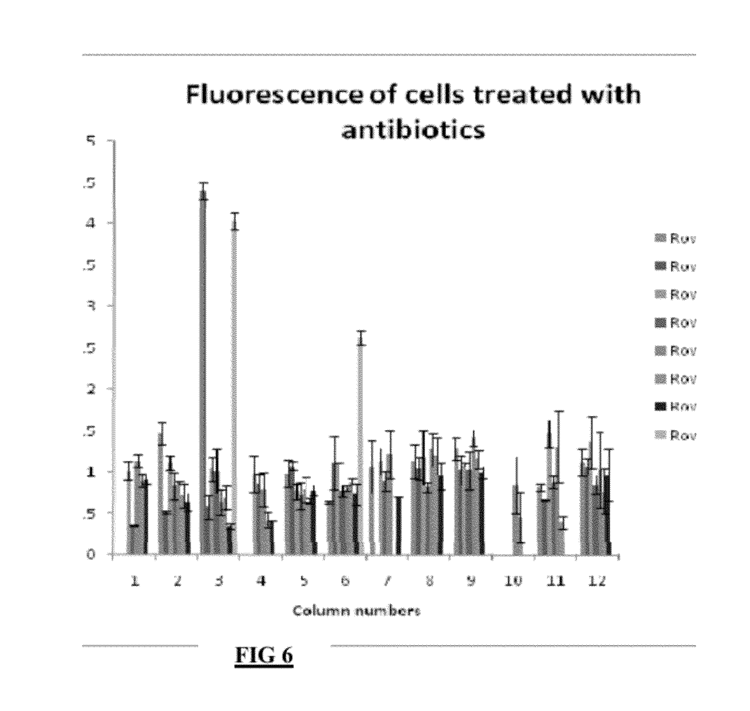

Cells grown with sub-bacteriostatic concentration of cerulenin showed a fluorescence ratio of ˜4.5. Similarly, cells grown with thiolactomycin showed a ratio of ˜4.0 and cells grown with platensimycin showed a ratio of ˜2.6. Other cells maintained a fluorescence ratio of ˜1. Visualization of the data by bar graph (FIG. 6) indicates that the fatty acid biosynthesis inhibitors, cerulenin, thiolactomycin, and platensimycin, can be detected in this high-throughput screen. Bischloroanthrabenzoxocinone was not detected in this assay probably because of its known poor cell permeability (Ref 10).

### Example 4

### Identification of Elongated Fatty Acids in the B392 Cell Lipid Biosynthetic Machinery from the Azido Fatty Acid (C₆N₃) Fed to the Cells

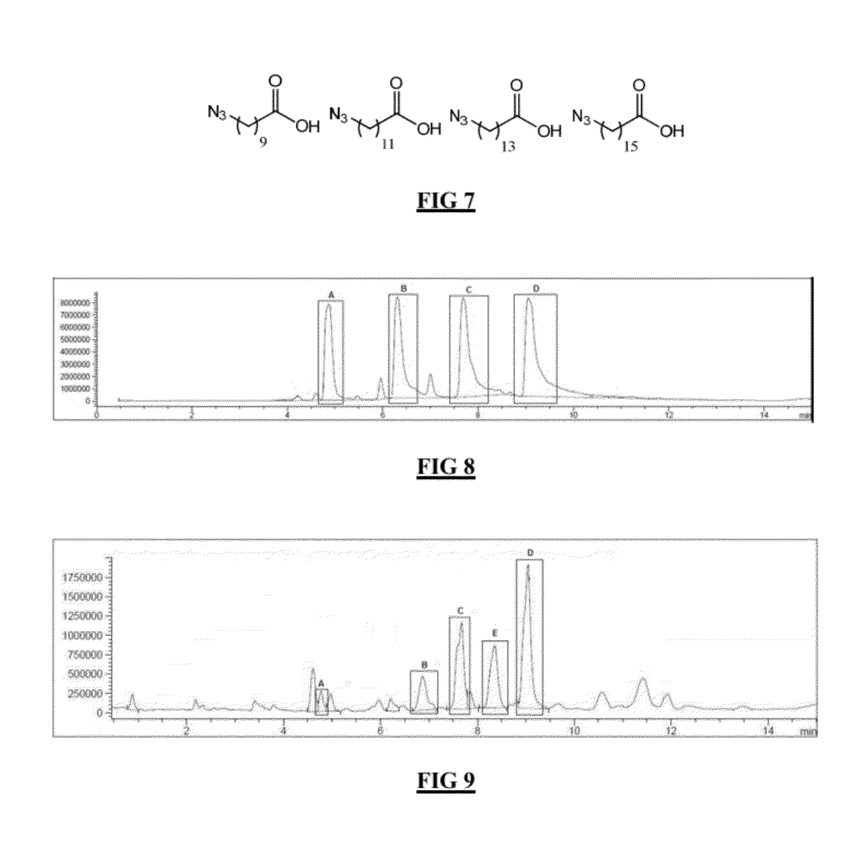

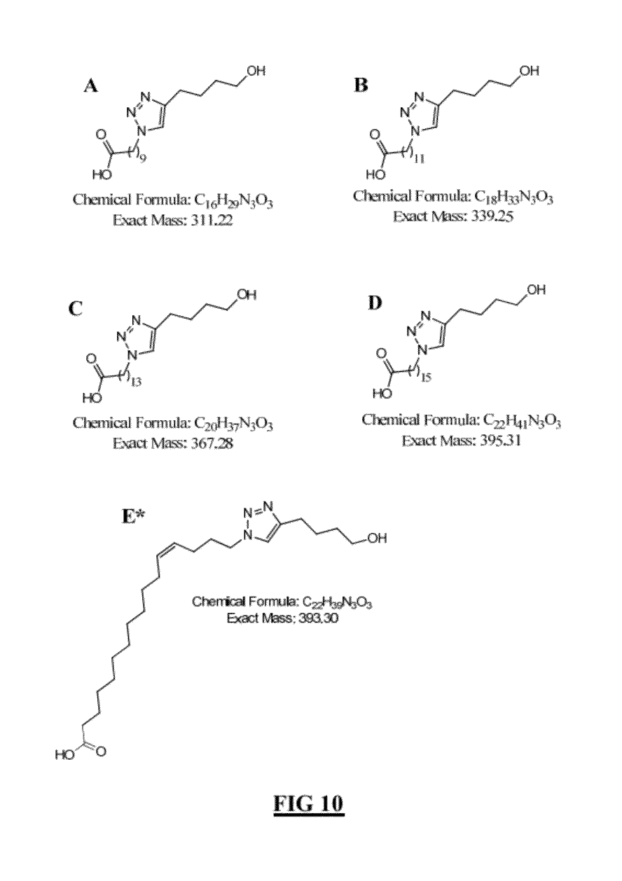

In this example, elongation of C₆N₃ by the B392 cells lipid biosynthetic machinery was confirmed by identification of some elongation products. The most common elongation products expected to be found within cells fed with short and medium chain co-azido fatty acid were the 10-carbon, 12-carbon, 14-carbon, and 16-carbon azido fatty acid analogues (FIG. 7 and Ref. 9). These long chain ω-azido fatty acids were prepared and reacted with 5-hexyn-1-ol to form analytical standards that are easily separable on reverse phase HPLC and contain triazoles that absorb at 230 nm (FIG. 10). An HPLC-UV-MS of a 1:1:1:1 molar mixture of the four fatty acid triazole derivatives resulted in four distinguishable peaks (FIG. 8). Then, a saponified lipid extract of V. harveyi B392 cells fed with 5 mM C₆N₃ for 4 hrs was “click reacted” in the same fashion and analyzed by HPLC-UV-MS (FIG. 9).

In particular, the analytical standards were prepared as follows: the azido analogues (2.2 mmol) were treated with 5-hexyn-1-ol (2.6 mmol) in the presence of copper acetate (0.2 mmol) and sodium ascorbate (0.4 mmol) in tetrahydrofuran (20 mL) under reflux overnight to form desired triazole derivatives. The products were purified by silica gel chromatography, with washes of 60-80% ethyl acetate in hexane at 10% increments and using 10% methanol in dichloromethane as the eluent. The solvent was evaporated using rotovap and any residual solvent was removed using a high vacuum pump. For HPLC-UV-MS analysis, 5 mM of each sample was dissolved in 5% methanol in dichloromethane.

In particular, the click reaction of cellular lipid extract was performed as follow: the cell culture was transferred to centrifuge tubes and spun at 6000 g for 15 minutes to collect cells in a pellet. The supernatant was removed and any non-incorporated C₆N₃ within the cell pellet were washed out by re-suspending the pellet in 0.9% NaCl and centrifuging at 6000 g for 15 minutes twice. The cell pellet was suspended in 80 mL of a 1:2:0.8 mixture of chloroform, methanol, and water (Refs. 12 and 13). The sample was sonicated to lyse the cells, and the mixture was left overnight. More water and chloroform was added to the mixture to create a biphasic system. The mixture was poured into an extraction funnel, and the chloroform phase was collected in a flask. The chloroform was evaporated with the rotovap, and potassium hydroxide (1M) and 95% ethanol (2 mL) were added. The potassium hydroxide was allowed to hydrolyze the lipids under reflux at 100° C. for one hour. Afterwards, 5 mL of water was added, then, 5 mL of a 1:1 mixture of hexane and diethyl ether were added and the hexane/diethyl ether mixture was collected after mixing and phase separation. This was repeated twice more. The pH of the water layer was then lowered to 2 using 1M HCl. Then, 5 mL of the 1:1 mixture of hexane and diethyl ether was added to the water layer, mixed, and collected for a total of three times. The hexane/diethyl ether mixture was evaporated using the rotovap and the lipid products were retrieved. These products were subsequently “click reacted,” under the conditions mentioned above with the assumption that 100% of the azido fatty acids were incorporated. Then the products were passed through a silica gel column using the same elution conditions as for the analytical standards, and analyzed by HPLC-UV-MS.

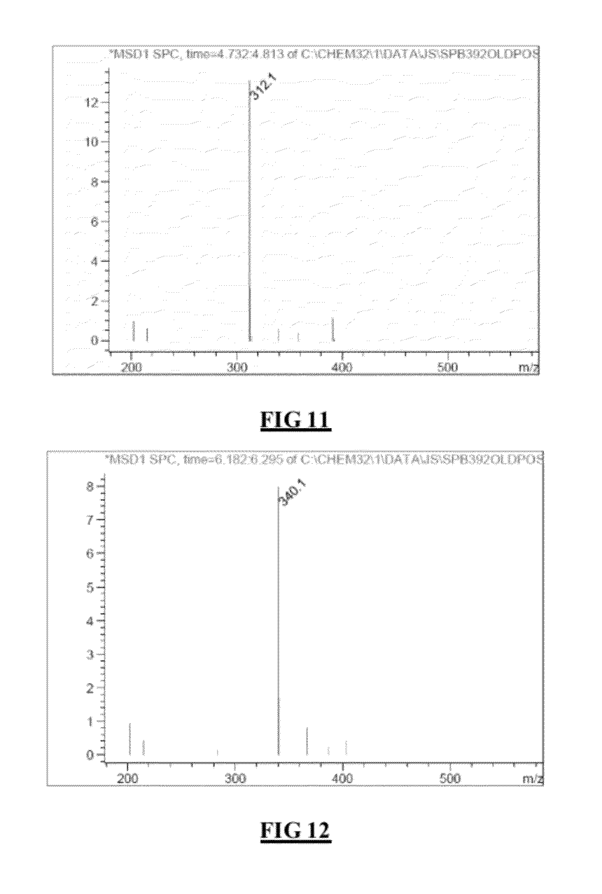

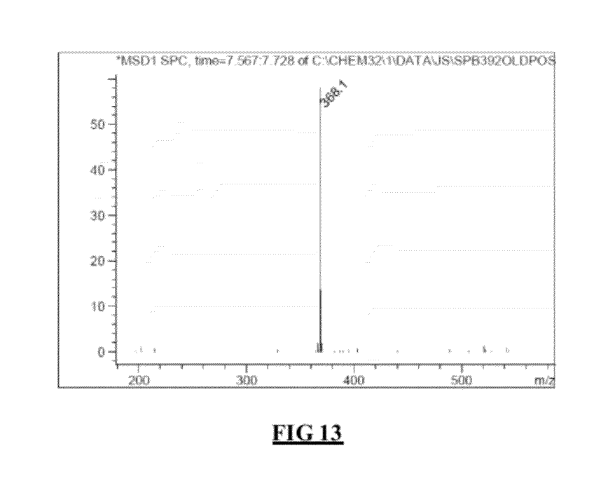

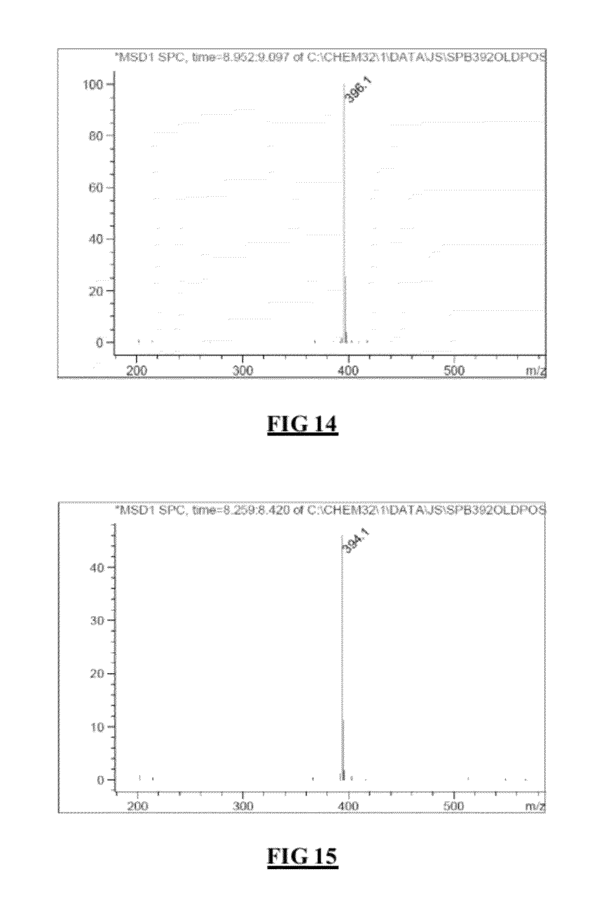

The data showed successful detection of the 10-carbon, 12-carbon, 14-carbon, and 16-carbon saturated fatty acid analogues in the V. harveyi B392 lipid extract (FIGS. 9-15). In addition, a substantial peak corresponding to the 16-carbon unsaturated fatty acid triazole derivative could be observed. This finding indicated that C₆N₃ was not only being elongated by the cellular machinery of V. harveyi B392, but also being desaturated.

### Example 5

### An Expected Method of High-Throughput Assay to Identify Various Effective Concentrations of One or More Fatty Acid Biosynthesis Inhibitors

In this example, a high-throughput cell-based antibiotic screen, which can be used to identify compounds capable of altering fatty acid biosynthesis is described. B392 cells in a well plate can be treated with various concentrations of one or more compounds capable of altering a fatty acid biosynthesis.

TABLE 4

| Candidate Cell | Concentration |
|---|---|
| Compound 1 | C1 |
| Compound 1 | C2 |
| Compound 1 | C3 |
| Compound 1 | C4 |
| Compound 1 | C5 |
| Compound 2 | C1 |
| Compound 2 | C2 |
| Compound 2 | C3 |
| Compound 2 | C4 |
| Compound 2 | C5 |

B392 cells given no drug treatment can serve as controls. Cells can be fed with C₆N₃ and allowed to grow overnight. Extracellular C₆N₃ can be washed away with 0.9% NaCl, and cellular lipids can be click-reacted with 50 μM cyclooctyne-coumarin conjugate for 30 minutes at 37° C. Non-reacted cyclooctyne-coumarin conjugate can be washed away with 0.9% NaCl, and cells re-suspended in 0.9% NaCl to be analyzed by plate reader. Any cells grown in cultures containing concentration of compounds capable of interfering with fatty acid biosynthesis by way of inhibition or enhancement would have significantly lower fluorescence or significantly higher fluorescence, respectively in comparison to non-treated cells due to decreased or increased incorporation of C₆N₃, respectively.

In particular, B392 cells can be grown in 150 mL cultures of synthetic media (Ref. 9) until OD 0.3 is reached. Cells can be transferred (1 mL of the culture loaded into each well) to a 2 mL 96-well plate via a multi-pipette robot (this robot can be used for most liquid transfers mentioned in this example). To each well, 5 μL of antibiotic from a 200× stock antibiotic plate in DMSO can be introduced to create final treatment of concentrations of 500 μM, 50 μM, and 5 μM. After 30 minutes, 10 μL of 500 mM C₆N₃ in DMSO can be introduced to each well for a final treatment concentration of 5 mM. Cells can then be grown overnight and then spun down at 2000 g for 10 minutes. Supernatant can be removed, and then non-incorporated C₆N₃ washed from cells three times by re-suspending the cells in 1 mL of 0.9% NaCl, spinning the cells down at 2000 g for 10 minutes, and removing the supernatant. The cells can then be re-suspended in 200 μL of 50 μM cyclooctyne-coumarin conjugate in 0.9% NaCl and incubated at 37° C. for 30 minutes. Cells can then be collected and washed three more times with 1 mL of 0.9% NaCl to remove non-reacted cyclooctyne-coumarin conjugate, and cells re-suspended in 400 μL of 0.9% NaCl can be analyzed by plate reader for fluorescence (excitation 380 nm, detection 500 nm) and cell density.

When considering the fluorescence ratio (see explanation of TABLES 1 and 2 in Example 1) between non-treated cells and drug treated cells, cells treated with compounds capable of altering a fatty acid biosynthesis would have a fluorescence ratio much greater than 1, while cells treated with fatty acid biosynthesis enhancing compounds would have a fluorescence ratio much less than 1.

### Example 6

### An Expected Method of a High-Throughput Assay to Identify Various Cells Capable of Elongating Exogenous Fatty Acids

In this example, a high-throughput cell-based antibiotic screen, which can be used to identify capable of elongating exogenous fatty acids is described.

TABLE 5
Candidate Cell

Candidate Cell 1
Candidate Cell 2
Candidate Cell 3
Candidate Cell 4
Candidate Cell 5
Candidate Cell 6
Candidate Cell 7
Candidate Cell 8
Candidate Cell 9
Candidate Cell 10

Cells known to elongate exogenous fatty acids (e.g. V. harveyi B392 cells) can be used as controls. Cells can be fed with C₆N₃ and allowed to grow overnight. Extracellular C₆N₃ can be washed away with 0.9% NaCl, and cellular lipids can be click-reacted with 50 μM cyclooctyne-coumarin conjugate for 30 minutes at 37° C. Non-reacted cyclooctyne-coumarin conjugate can be washed away with 0.9% NaCl, and cells re-suspended in 0.9% NaCl to be analyzed by plate reader. Any capable of elongating exogenous fatty acids would have significantly higher fluorescence in comparison cells which are not capable of elongating fatty acids, due to an increased incorporation of C₆N₃ in the former.

In particular, cells can be grown in 150 mL cultures of synthetic media (Ref. 9) until OD 0.3 is reached. Cells can be transferred (1 mL of the culture loaded into each well) to a 2 mL 96-well plate via a multi-pipette robot (this robot can be used for most liquid transfers mentioned in this example). To each well, 5 μL of antibiotic from a 200× stock antibiotic plate in DMSO can be introduced to create final treatment of concentrations of 500 μM, 50 μM and 5 μM. After 30 minutes, 10 μL of 500 mM C₆N₃ in DMSO can be introduced to each well for a final treatment concentration of 5 mM. Cells can then be grown overnight and then spun down at 2000 g for 10 minutes. Supernatant can be removed, and then non-incorporated C₆N₃ washed from cells three times by re-suspending the cells in 1 mL of 0.9% NaCl, spinning the cells down at 2000 g for 10 minutes, and removing the supernatant. The cells can then be re-suspended in 200 of 50 μM cyclooctyne-coumarin conjugate in 0.9% NaCl and incubated at 37° C. for 30 minutes. Cells can then be collected and washed three more times with 1 mL of 0.9% NaCl to remove non-reacted cyclooctyne-coumarin conjugate, and cells re-suspended in 400 μL of 0.9% NaCl can be analyzed by plate reader for fluorescence (excitation 380 nm, detection 500 nm) and cell density.

### Example 7

### An Expected Method of a High-Throughput Assay to Identify Various Conditions Under which Cells are Capable of Elongating Exogenous Fatty Acids

In this example, a high-throughput cell-based antibiotic screen, which can be used to identify various conditions under which cells are capable of elongating exogenous fatty acids is described.

TABLE 6

| Condition 1 | Condition 2 |
|---|---|
| C1 of [labeled fatty acid precursor] | temperature (T1) during incubation |
| C2 of [labeled fatty acid precursor] | temperature (T1) during incubation |
| C3 of [labeled fatty acid precursor] | temperature (T1) during incubation |
| C4 of [labeled fatty acid precursor] | temperature (T1) during incubation |
| C5 of [labeled fatty acid precursor] | temperature (T1) during incubation |
| C6 of [labeled fatty acid precursor] | temperature (T2) during incubation |
| C6 of [labeled fatty acid precursor] | temperature (T3) during incubation |
| C6 of [labeled fatty acid precursor] | temperature (T5) during incubation |
| C6 of [labeled fatty acid precursor] | temperature (T5) during incubation |
| C6 of [labeled fatty acid precursor] | temperature (T6) during incubation |

A cell known to elongate exogenous fatty acids under a condition known to allow the cell to elongate exogenous fatty acids can be used as a control. One or more conditions can separately vary in various samples. For example, temperature during incubation and/or concentration of labeled fatty acid precursor can each be varied while the other remains constant and any other variables remain constant (See TABLE 6, C1=concentration 1, T1=temperature 1, etc.) Cells can be fed with C₆N₃ and allowed to grow overnight. Extracellular C₆N₃ can be washed away with 0.9% NaCl, and cellular lipids can be click-reacted with 50 μM cyclooctyne-coumarin conjugate for 30 minutes at 37° C. Non-reacted cyclooctyne-coumarin conjugate can be washed away with 0.9% NaCl, and cells re-suspended in 0.9% NaCl to be analyzed by plate reader. Any condition suitable for elongating exogenous fatty acids would have significantly higher fluorescence in comparison to conditions which are not suitable for elongating fatty acids, due to an increased incorporation of C₆N₃ in the former.

In particular, cells can be grown in 150 mL cultures of synthetic media (Ref. 9) until OD 0.3 is reached. Cells can be transferred (1 mL of the culture loaded into each well) to a 2 mL 96-well plate via a multi-pipette robot (this robot can be used for most liquid transfers mentioned in this example). To each well, 5 μL of antibiotic from a 200× stock antibiotic plate in DMSO can be introduced to create final treatment of concentrations of 500 μM, 50 μM and 5 μM. After 30 minutes, 10 μL of 500 mM C₆N₃ in DMSO can be introduced to each well for a final treatment concentration of 5 mM. Cells can then be grown overnight and then spun down at 2000 g for 10 minutes. Supernatant can be removed, and then non-incorporated C₆N₃ washed from cells three times by re-suspending the cells in 1 mL of 0.9% NaCl, spinning the cells down at 2000 g for 10 minutes, and removing the supernatant. The cells can then be re-suspended in 200 μL of 50 μM cyclooctyne-coumarin conjugate in 0.9% NaCl and incubated at 37° C. for 30 minutes. Cells can then be collected and washed three more times with 1 mL of 0.9% NaCl to remove non-reacted cyclooctyne-coumarin conjugate, and cells re-suspended in 400 μL of 0.9% NaCl can be analyzed by plate reader for fluorescence (excitation 380 nm, detection 500 nm) and cell density.

Additional parameters that can be tested with methods herein described include media composition, temperature, introduction time point for a compound able to affect fatty acid elongation pathway and/or of other compound, and additional parameters identifiable by a skilled person.

### Example 8

### Qualitative Detection of Inhibition and Enhancement of Fatty Acid Elongation Pathways

The method and systems described in the application can be used to detect a signal related to a substance's ability to alter a fatty acid elongation pathway in both a qualitative and a quantitative manner, and also determine the degree to which a substance alters a fatty acid elongation pathway (e.g. the degree to which it inhibits or enhances a fatty acid elongation pathway).

FIG. 5 shows a schematic representation of a experiments settings directed to detect inhibition qualitatively. For example, Vibrio harveyi strain B392 can be treated with an exogenous N₃(CH₂)₅CO₂H(C₆N₃) as described in Example 1 and the azide group detected using a cyclooctyne-coumarin conjugate in a multi well plate such as the one illustrated in FIG. 5.

As can be seen from the schematic illustration of FIG. 5 the signal (in this case fluorescence) from the wells containing the candidate compounds being used in the method is compared to the signal from a well containing all system components except the candidate compound (that signal thus providing a baseline signal for unaltered fatty acid elongation). FIG. 5 additionally shows that after performance of the method, the ratio of the baseline signal to the observed signals will be greater than 1 in cases where the particular compound is an inhibitor of fatty acid elongation (due to lack of accumulation of the product of elongation of the labeled fatty acid precursor), and equal to 1 in cases where the fatty acid elongation pathway has not been altered at all by the candidate compound. A person of ordinary skill in the would also be able to tell that with candidate compounds that enhanced fatty acid elongation, the ratio of the baseline signal to the observed signal would be less than 1. In many cases, the signal can be measured qualitatively (for example, by visual inspection of the brightness or dimness of a particular observed fluorescence signal relative to the brightness of the baseline signal).

A skilled person would be able to understand that according to the experimental settings of FIG. 5 a detection of the fluorescence intensity can be performed to identify the extent of inhibition or enhancement of the fatty acid elongation pathway to provide a quantification of a fluorescence ratio with respect to the control thus providing a quantitative detection (see Example 9 below).

### Example 9

### Quantitative Detection of Inhibition and Enhancement of Fatty Acid Elongation Pathways and Related Determination of Degree of Inhibition or Enhancement of Fatty Acid Elongation Pathways

Experiments were performed where known inhibitors of fatty acid elongation pathway were tested on B392 cells and CY1723 with azide label precursor N₃(CH₂)₅CO₂H(C₆N₃) and the elongated fatty acid detected through flow cytometry as described in Example 2.

The results are illustrated in FIG. 4 which shows flow cytometry fluorescence curves detected following treatment of cells with different combination of inhibitors as illustrated in TABLE 2. The curves of FIG. 4 they show overlapping numerical values for the various detected signals. Based on the charts of FIG. 4 it is possible to determine a specific fluorescence value associated to each treatment and based on those values a distribution of the fluorescence intensities and the average fluorescence intensity. On this basis it is also possible to calculate a ratio of the baseline signal to the observed signals and verify whether this ratio is above or below 1 (see Example 8 above) as would be understood by a person skilled in the art.

A further example of this quantitative measurement can be seen in FIG. 6, which shows actual ratios of baseline signal to observed signal acquired in Example 3 above. In this particular case, degree of inhibition is shown where the tallest peaks indicate candidate compounds capable of the greatest amount of inhibition of fatty acid elongation, but degree of enhancement of some compounds is also shown in those peaks that show ratios below a value of 1.

A person of ordinary skill in the art would be able to realize that calculating the reciprocal of the above ratio (i.e. calculating the ratio of observed signal to baseline signal) would result in inhibitors of fatty acid elongation having ratios less than 1 and enhancers with ratios greater than 1 (the opposite of what was seen above). Not only would this continue to enable quantitative measurement of the signal (i.e. the degree of fatty acid elongation enhancement by measuring how far the reciprocal ratio is above 1), but it would also enable rapid qualitative determination of fatty acid elongation enhancers (by enabling rapid visual detection of tallest peaks), just as the previous ratio (baseline signal to observed signal) enabled rapid qualitative determination of inhibitors of fatty acid elongation by visual detection of the tallest peaks.

The examples set forth above are provided to give those of ordinary skill in the art a complete disclosure and description of how to make and use the embodiments of the compositions, arrangements, devices, compositions, systems and methods of the disclosure, and are not intended to limit the scope of what the inventors regard as their disclosure. All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by preferred embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified can be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein can be excluded from a claim of this disclosure. The disclosure illustratively described herein suitably can be practiced in the absence of any element or elements, limitation or limitations which is not specifically disclosed herein.

A number of embodiments of the disclosure have been described. The specific embodiments provided herein are examples of useful embodiments of the disclosure and it will be apparent to one skilled in the art that the disclosure can be carried out using a large number of variations of the devices, device components, methods steps set forth in the present description. As will be obvious to one of skill in the art, methods and devices useful for the present methods can include a large number of optional composition and processing elements and steps.

In particular, it will be understood that various modifications may be made without departing from the spirit and scope of the present disclosure. Accordingly, other embodiments are within the scope of the following claims.

### LIST OF REFERENCES

1. (a) Wright, H. T.; Reynolds, K. A. Antibacterial targets in fatty acid biosynthesis. Curr. Opin. Microbiol. 2007, 10, 447-453; (b) Zhang, Y.; White, W. S.; Rock, C. O. Inhibiting bacterial fatty acid synthesis. J. Biol. Chem. 2006, 281, 17541-17544.
2. (a) Young, K.; et al. Discovery of FabH/FabF inhibitors from natural products. Antimicrob. Agents Chemother. 2006, 50, 519-526; (b) Wang, J.; et al. Platensimycin is a selective FabF inhibitor with potent antibiotic properties. Nature 2006, 441, 358-361; (c) Singh, S. B.; Phillips, J. W.; Wang, J. Highly sensitive target-based whole-cell antibacterial discovery strategy by antisense RNA silencing. Curr. Opin. Drug Discov. & Dev. 2007, 10, 160-166; (d) Wang, J.; et al. Discovery of platencin, a dual FabF and FabH inhibitor with in vivo antibiotic properties. Proc. Nat. Acad. of Sci. USA 2007, 104, 7612-7616.
3. (a) Menendez, J. A.; Lupu, R. Fatty acid synthase and the lipogenic phenotype in cancer pathogenesis. Nat. Rev. Cancer 2007, 7, 763-777; (b) Kuhajda, F. P. Fatty acid synthase and cancer: new application of an old pathway. Cancer Res. 2006, 66, 5977-5980; (c) Kuhajda, F. P. Fatty-acid synthase and human cancer. Nutrition 2000, 16, 202-208.
4. Wenk, M. R. The emerging field of lipidomics. Nat. Rev. Drug Discovery 2005, 4, 594-610.
5. Sletten, E. M.; Bertozzi, C. R. Bioorthogonal chemistry: fishing for selectivity in a sea of functionality. Angew. Chem. Int. Ed. 2009, 48, 6974-6998.
6. (a) Jewetta, J. C.; Bertozzi, C. R. Cu-free click cycloaddition reactions in chemical biology. Chem. Soc. Rev., 2010, 39, 1272-1279; (b) Beatty, K. E.; Fisk, J. D.; Smart, B.; Lu, Y. Y.; Szychowski, J.; Hangauer, M. J.; Baskin, J. M.; Bertozzi, C. R.; Tirrell, D. A. Live cell imaging of cellular proteins via a strain-promoted azide-alkyne cycloaddition. ChemBioChem, 2010, 11, 2092-2095; (c) Beatty, K. E.; Szychowski, J.; Fisk, J. D.; Tirrell, D. A. A bodipy-cyclooctyne for proteomic imaging in live cells. ChemBioChem, 2011, 12, 2137-2139.
7. Jang, Y.; Chan, C. H.; Cronan, J. E. The soluble acyl-acyl carrier protein synthetase of Vibrio harveyi B392 is a member of the medium chain acyl-CoA synthetase family. Biochemistry 2006, 45, 10008-10019.
8. (a) Matsumae, A.; Nomura, S.; Hata, T. Studies on cerulenin. IV. Biological characteristics of cerulenin. J. Antibiot. 1964, 17, 1-7; (b) Awaya, J.; Omura S. Substitution of cellular fatty acids in yeast cells by the antibiotic cerulenin and exogenous fatty acids. Biochim. Biophys. Acta 1975, 409, 267-273.
9. Byers, D. M. Elongation of exogenous fatty acids by the bioluminescent bacterium Vibrio harveyi. J. Bacteriol. 1989, 171, 59-64.
10. Kodali, S. et al. Determination of selectivity and efficacy of fatty acid synthesis inhibitors. J. Biol. Chem. 2005, 280, 1669-1677.
11. Noto, T.; Miyakawa, S.; Oishi, H.; Endo, H.; Okazaki, H. Thiolactomycin, a new antibiotic. III. In vitro antibacterial activity. J. Antibiot. 1982, 35, 401-410.
12. Lewis, T.; Nichols, P D.; McMeekin T. A. Evaluation of extraction methods for recovery of fatty acids from lipid-producing microheterotrophs. J. Microbiol. Methods. 2000, 43, 107-116
13. Christie, W. W. Lipid Analysis: Isolation, Separation, Identification and Structural Analysis of Lipids Third Edition; The Oily Press: Bridgwater, 2003; 205-206.

1. A method to identify a compound capable of altering a fatty acid elongation pathway, the method comprising:
contacting a candidate compound and a fatty acid precursor comprising a label with a cell comprising the enzymes required in the fatty acid elongation pathway, the contacting performed for a time and under condition to allow elongation of the fatty acid precursor comprising the label through the fatty acid elongation pathway and to allow interference of the candidate compound with the fatty acid elongation pathway; and
detecting the label following the contacting.

2. The method of claim 1, further comprising removing the fatty acid precursor comprising the label before the detecting.

3. The method of claim 1, wherein the detecting is performed by detecting quantitatively a labeling signal associated to the label.

4. The method of claim 1, wherein the detecting is performed by detecting qualitatively a labeling signal associated to the label.

5. The method of claim 3, wherein the labeling signal is a luminescent signal selected from the group consisting of fluorescent, phosphorescent, chemiluminescent, and bioluminescent.

6. The method of claim 4, wherein the labeling signal is a luminescent signal selected from the group consisting of fluorescent, phosphorescent, chemiluminescent, and bioluminescent.

7. The method of claim 1, wherein the detecting is performed by:
detecting a labeling signal associated to the label and comparing the detected labeling signal to a reference signal associated to the fatty acid elongation pathway in the cell in absence of the candidate compound.

8. The method of claim 1, wherein the label is a functional group capable of binding to a bioorthogonal functional group in a probe emitting a labeling signal, and wherein the detecting is performed by treating the cell with the probe and detecting the labeling signal following the treating.

9. The method of claim 8, further comprising removing the probe before detecting the labeling signal.

10. The method of claim 8, wherein the label is an azide functional group, the bioorthogonal functional group is an alkyne.

11. The method of claim 8, wherein the labeling signal is a luminescent labeling.

12. The method of claim 1, wherein the fatty acid precursor comprises a saturated or unsaturated aliphatic chain comprising approximately 2 to 10 carbons.

13. The method of claim 1, wherein the fatty acid precursor comprising the label has a formula: N₃(CH₂)ₙCOOH, wherein n ranges from approximately 1 to 9.

14. The method of claim 1, wherein the cell is a bacterial cell.

15. A method to determine an effective concentration of one or more compounds capable of altering a fatty acid elongation pathway, the method comprising:
contacting the one or more compounds at a first concentration and a labeled fatty acid precursor with a cell comprising the enzymes required in the fatty acid elongation pathway, the contacting performed for a time and under condition to allow a first elongation of the labeled fatty acid precursor through the fatty acid elongation pathway and to allow interference of the one or more compound at the first concentration with the fatty acid elongation pathway;
detecting a first labeling signal associated with the labeled fatty acid precursor following the first elongation, the first labeling signal associated with the first concentration, the detecting performed to determine concentration effective for altering a fatty acid elongation pathway.

16. The method of claim 15, further comprising contacting the one or more compounds capable of altering the fatty acid elongation pathway each at one or more second concentrations, and a labeled fatty acid precursor with the cell comprising the enzymes required in the fatty acid elongation pathway, the contacting performed for a time and under condition to allow one or more second elongations of the labeled fatty acid precursor through the fatty acid elongation pathway and to allow interference of the compound at each of the one or more second concentrations with the fatty acid elongation pathway;
detecting one or more second labeling signals associated with the labeled fatty acid precursor following the one or more second elongations each labeling signal associated with each one or more second concentrations; and
comparing the first labeling signal with the one or more second labeling signals to determine one or more concentrations effective for altering a fatty acid elongation pathway.

17. The method of claim 15, wherein the compound capable of altering the fatty acid elongation pathway is an inhibitor of the fatty acid elongation pathway.

18. The method of claim 15, wherein the compound capable of altering the fatty acid elongation pathway is an enhancer of the fatty acid elongation pathway.

19. The method of claim 15, wherein the compound capable of altering the fatty acid elongation pathway is an antibiotic.

20. A method to identify a cell of capable of elongating an exogenous fatty acid, the method comprising:
contacting a candidate cell and a fatty acid precursor comprising a label, the contacting performed for a time and under condition to allow elongation of the labeled fatty acid precursor through the fatty acid elongation pathway; and
detecting the label following the contacting.

21. The method of claim 20, wherein the detecting is performed through detection of a labeling signal and detection of the labeling signal indicates that the candidate cell comprises the enzymes required in the metabolic pathway of fatty acid elongation.

22. A method for identifying a compound capable of inducing apoptosis in a cell, the method comprising:
identifying a compound capable of altering a fatty acid elongation pathway in the cell with the method of claim 1, wherein the identified compound is a compound capable of inhibiting the fatty acid elongation pathway; and
contacting the identified compound with the cell for a time and under conditions to allow interference of the compound with the fatty acid elongation pathway;
detecting viability of the cell following the contacting.

23. A method to identify a value or a range of values of a parameter associated with ability of a cell of elongating an exogenous fatty acid, the method comprising:
contacting the cell and a fatty acid precursor comprising a label, the contacting performed for a time and under condition to allow elongation of the labeled fatty acid precursor through the fatty acid elongation pathway, the conditions comprising a first value of the parameter;
detecting a first labeling signal from the label, the first labeling signal associated with the first value of the parameter, the detecting performed to determine the value of the parameter associated with ability of the cell of elongating the exogenous fatty acid.

24. The method of claim 23, further comprising:
contacting the cell at one or more second values of the parameter and a fatty acid precursor comprising a label, the contacting performed for a time and under conditions to allow elongation of the labeled fatty acid precursor through the fatty acid elongation pathway, the conditions comprising a one or more second values of the parameter;
detecting one or more second labeling signal from the label, each of the one or more second labeling signal associated with the each of the one or more second values of the parameter; and
comparing the first labeling signal with the one or more labeling signals to determine one or more values associated with ability of the cell of elongating the exogenous fatty acid.

25. The method of claim 23, wherein the contacting is performed by contacting the precursor with a cell and a compound capable of altering the fatty acid elongation pathway.

26. The method of claim 23, wherein the parameter is a concentration of fatty acid precursor under which the contacting is performed.

27. The method of claim 23, wherein the parameter is a temperature at which the contacting is formed.

28. The method of claim 23, wherein the parameter is a number of carbons in the labeled fatty precursor.
